(19) United States
(12) Patent Application Publication
(10) Pub. No.: US 2025/0275918 A1
(43) Pub. Date: Sep. 4, 2025

(54) LIPID COMPOSITIONS AND METHODS FOR NUCLEIC ACID DELIVERY

(71) Applicant: GREENLIGHT BIOSCIENCES, INC., Medford, MA (US)

(72) Inventors: Mohammad Parvez ALAM, Medford, MA (US); Lorenzo AULISA, Medford, MA (US); Jeffrey HUGHES, Medford, MA (US); Mohit GUPTA, Medford, MA (US); Saikat MANNA, Medford, MA (US)

(21) Appl. No.: 18/857,934

(22) PCT Filed: Apr. 21, 2023
(86) PCT No.: PCT/US2023/019411

§ 371 Date: Oct. 18, 2024

Related U.S. Application Data
(60) Provisional application No. 63/333,153, filed on Apr. 21, 2022.

(51) Int. Cl. A61K 9/1271; A61K 9/00; A61K 9/51

(52) U.S. Cl. CPC A61K 9/1271; A61K 9/0019; A61K 9/5123

(57) ABSTRACT

The present invention relates to ionizable lipids and lipid nanoparticle compositions thereof. The nanoparticle compositions are useful in the delivery of therapeutic agents such as nucleic acids.

This application claims the benefit of and priority to U.S. Provisional Application No. 63/333,153, filed on Apr. 21, 2022, which is hereby incorporated herein by reference in its entirety.

### BACKGROUND OF THE INVENTION

The effective delivery of biologically active substances such as messenger RNA (mRNA) represents a continuing challenge. mRNA has significant therapeutic potential, but efficiency of delivery to target cells remains challenging. In particular, the delivery of nucleic acids such as RNA to cells is made difficult by its relative instability and low cell permeability. Currently available delivery methods and nanoparticle compositions cannot deliver more than 1% of the loaded mRNA. Thus, there exists a need to develop new lipids and compositions thereof to facilitate the delivery of biologically active substances, such as nucleic acids, to cells.

### SUMMARY OF THE INVENTION

In various aspects and embodiments, the present invention provides lipid nanoparticle compositions comprising an ionizable lipid compound having a plurality of nitrogen atoms in a main chain together with lipophilic substituents as described in detail herein. In some embodiments, lipophilic substituents are selected to allow for interactions with encapsulated nucleic acid, such as through cyclic and/or substantially planar moieties. Such ionizable lipid compounds provide advantages for nucleic acid delivery to cells, including but not limited to RNA delivery (e.g., mRNA delivery).

In various embodiments, the lipid nanoparticle composition comprises an ionizable lipid of Formula (I):

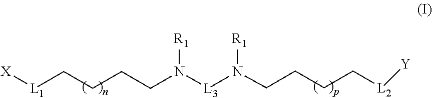

wherein:
each R₁ independently is H or a substituent;
L₁ is —OC(═O)— or —C(═O)O—;
L₂ is —OC(═O)— or —C(═O)O—;
L₃ is selected from the group consisting of:

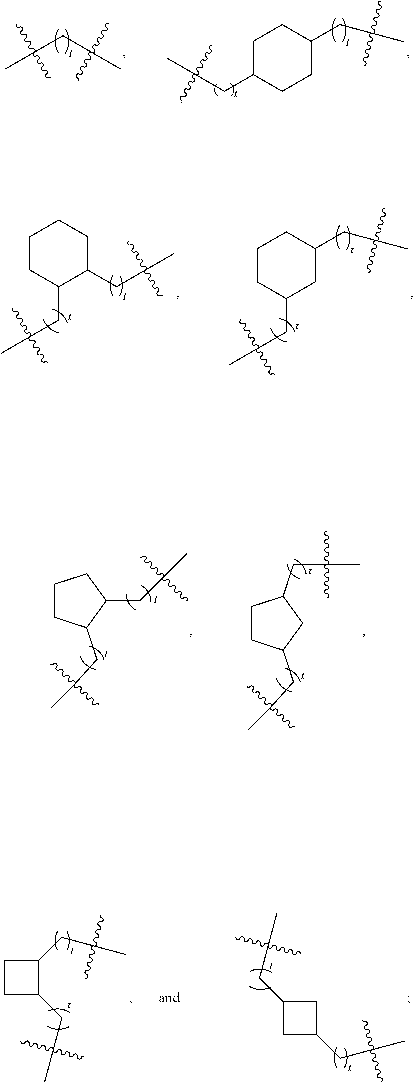

each of n, t, and p is independently selected from 0, 1, 2, 3, 4, 5, 6, 7, 8, 9, and 10;
and X and Y are each independently selected lipophilic moieties having at least six carbon atoms.

In various embodiments, L₃ is a bi-cyclic head group selected from the group consisting of

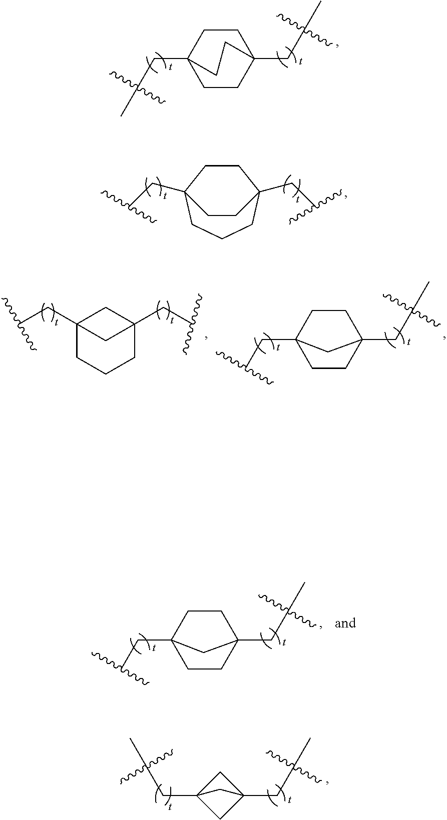

wherein t is 0, 1, 2, 3, 4, 5, 6, 7, 8, 9, and 10; when t is 0, the two N atoms are connected to the bicycle ring directly.

In various embodiments, each R₁ is independently selected from: H or a substituent such as (C₁-C₆)alkyl, substituted (C₁-C₆)alkyl, (C₁-C₆)alkenyl, substituted (C₁-C₆)alkenyl, cycloalkyl, cycloalkenyl, aryl, heteroaryl, and heterocyclo.

In some embodiments, each R₁ is independently selected from (halo)(C₁-C₆)alkyl, (alkoxy)(C₁-C₆)alkyl, (hydroxy)(C₁-C₆)alkyl, —(CH₂)ₙ—S—(C₁-C₆)alkyl, —(CH₂)ₙ—O—(C₁-C₆)alkyl, —C(O)(C₁-C₆)alkyl, (C₃-C₁₂)cycloalkyl, and (C₃-C₁₂)cycloalkenyl, any of which is optionally independently substituted as allowed by valence.

In some embodiments, each R₁ is independently selected from —(CH₂)ₙCHZR′, —CHZR′, —CZ(R′)₂, and —(CH₂)ₙZ, wherein Z is selected from —OC(O)(C₁-C₆)alkyl, —C(O)O(C₁-C₆)alkyl, —OC(O)(C₁-C₆)alkenyl, —C(O)O(C₁-C₆)alkenyl, (C₃-C₁₂)cycloalkyl, and (C₃-C₁₂)cycloalkenyl, any of which is optionally independently substituted as allowed by valence. R′ at each occurrence is a substituent, such as a substituent independently selected from halo, hydroxyl, cyano, nitro, oxo, (C₁-C₆)alkyl, (C₁-C₆)alkoxy, (C₁-C₆) haloalkyl, (C₂-C₆)alkenyl, (C₁-C₆)alkynyl, (C₃-C₁₂)cycloalkyl, (C₃-C₁₂)cycloalkenyl, heterocyclo, aryl, and heteroaryl.

In certain embodiments, each R₁ is independently a C₁ to C₃ alkyl, and which is optionally methyl, ethyl or isopropyl.

In certain embodiments, each R₁ is independently selected from the group consisting of

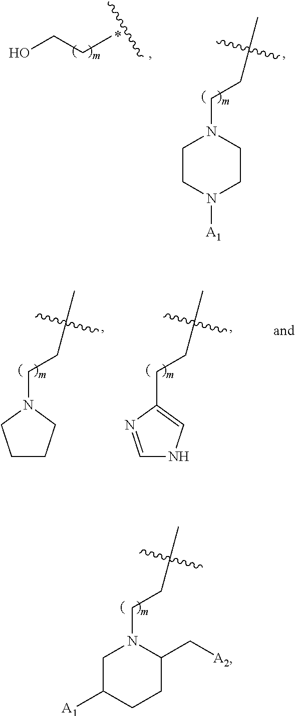

wherein m is 1, 2, 3, 4, 5, 6, 7, 8, 9, or 10; A₁ or A₂ is H, C₁ to C₃ alkyl, and which is optionally methyl, ethyl or isopropyl.

In still other embodiments, each R₁ is independently —(CH₂)ₙZ, and Z is aryl or heterocyclo. For example, each R₁ independently can be —(CH₂)ₙZ, where each Z is selected from phenyl, morpholinyl, pyrrolidinyl, imidazolidinyl, imidazolyl, pyrazolidinyl, pyrazolyl, oxazolidinyl, oxazolyl, pyridinyl, piperidinyl, diazinanyl, and diazinyl, and which is optionally substituted (e.g., with one or more substituents).

In various embodiments of Formula I, X and Y are lipophilic moieties, which can be independently selected from linear or branched alkyl, linear or branched alkenyl, sterol, polyphenol, flavonoid, and tocopherol. For example, in some embodiments one or both of X and Y are cholesteryl moiety. In some embodiments, one or both of X and Y are ester of tocopherol, which is optionally alpha, beta, gamma, or delta tocopherol. In embodiments, one or both of X and Y are a branched alkyl or alkenyl having from 8 to 30 carbon atoms. In some embodiments, one or both of X and Y is/are:

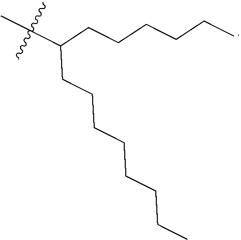 [SMILES: *C(CCCCCC)CCCCCCCC]

In various embodiments, wherein X and/or Y are a flavonoid, the flavonoid is optionally selected from quercetin, rutin, macluraxanthone, genistein, scopoletin, daidzein, taxifolin, naringenin, abyssinones, eriodictyol, fisetin, theaflavin, peonidin, diosmetin, tricin, biochanin, hesperidin, epicatechin, myricetin, kaempferol, luteolin, and apigenin.

In various embodiments of the compound of Formula I, t is an integer from 2 to 10. In some embodiments, t is 3, 4, 5, 6, 7, or 8.

In various embodiments of the compounds of Formula I, n and p (which can be the same or different) are each an integer in the range of 3 to 10. In some embodiments, n and p (which can be the same or different) are selected from 6, 7, and 8. In some embodiments, n and p are the same.

In various embodiments, the lipid nanoparticle composition comprises an ionizable lipid of Formula (II), (III) or (IV), wherein n, t. p. L₁, L₂, and R₁ are defined as in Formula (I).

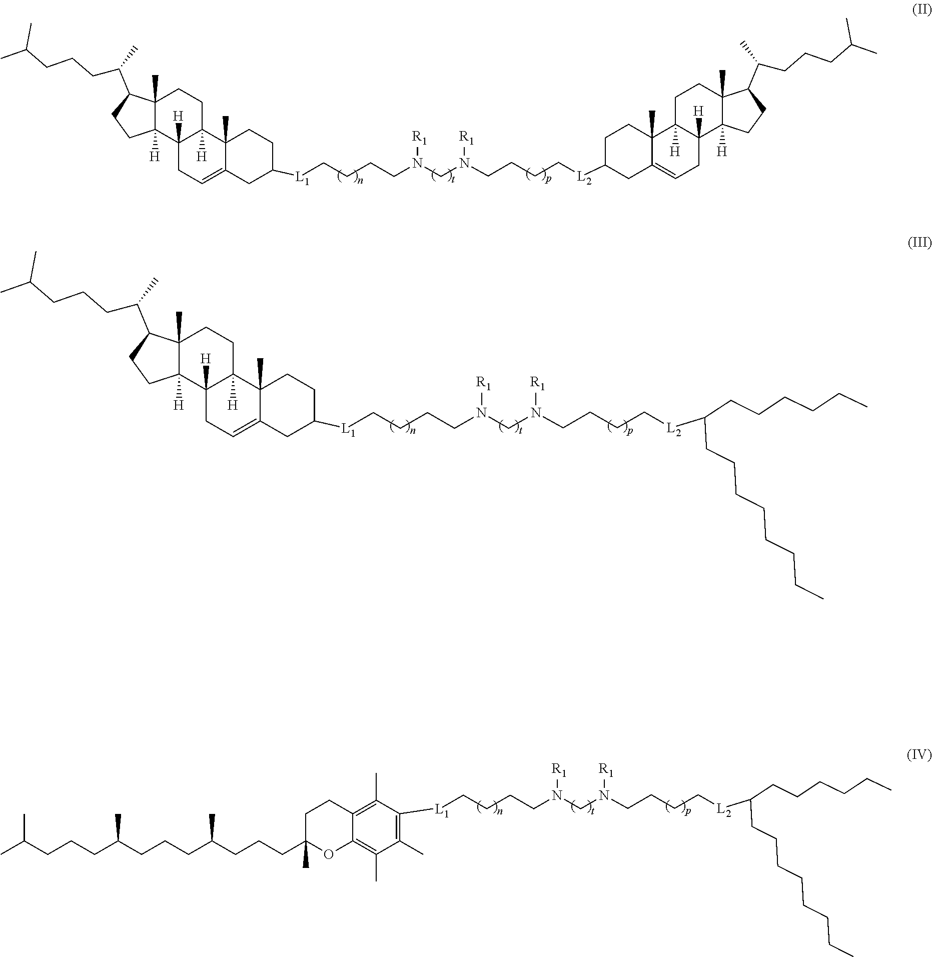

In various embodiments, the composition comprises an ionizable lipid shown in Table 1. Such compounds can be made according to the methods and scheme described in Example 1. Thus, the ionizable lipid of Formula 1 may be selected from:
(propane-1,3-diylbis(methylazanediyl))bis(heptane-7,1-diyl)bis(2-hexyldecanoate),
(propane-1,3-diylbis(ethylazanediyl))bis(heptane-7,1-diyl)bis(2-hexyldecanoate),
(octane-1,8-diylbis(methylazanediyl))bis(heptane-7,1-diyl)bis(2-hexyldecanoate),
(ethane-1,2-diylbis(benzylazanediyl))bis(heptane-7,1-diyl)bis(2-hexyldecanoate),
((((1R,3S)-cyclohexane-1,3-diyl)bis(methylene))bis(azanediyl))bis(heptane-7,1-diyl)bis(2-hexyldecanoate),
(((1s,4s)-cyclohexane-1,4-diyl)bis(azanediyl)) bis(heptane-7,1-diyl)bis(2-hexyldecanoate),
(cyclohexane-1,2-diylbis(azanediyl))bis(heptane-7,1-diyl)bis(2-hexyldecanoate),
(((1R,2S)-cyclohexane-1,2-diyl)bis(methylazanediyl))bis(heptane-7,1-diyl)bis(2-hexyldecanoate),
(propane-1,3-diylbis(isopropylazanediyl))bis(heptane-7,1-diyl)bis(2-hexyldecanoate),
(ethane-1,2-diylbis(tert-butylazanediyl))bis(heptane-7,1-diyl)bis(2-hexyldecanoate), and
(bicyclo[1.1.1]pentane-1,3-diylbis(methylazanediyl))bis(heptane-7,1-diyl)bis(2-hexyldecanoate)

In some embodiments, the lipid nanoparticles, e.g., for encapsulating nucleic acid such as mRNA, may comprise: a cationic or ionizable lipid of Formula I, a neutral lipid, a structural lipid, and a PEGylated lipid, or may be formulated according to other nanoparticle formulations known in the art. In various embodiments, the largest dimension of the lipid nanoparticles is about 200 nm or less. In exemplary embodiments, the mean diameter of the lipid nanoparticles is in the range of about 50 nm to about 125 nm (e.g., in the range of about 60 to 110 nm).

In various embodiments, the lipid nanoparticles in the composition encapsulate one or more therapeutic, prophylactic, or diagnostic agents. For example, the lipid nanoparticles may encompass one or more therapeutic proteins. For example, the lipid nanoparticles can encapsulate one or more polynucleotides, which can be DNA (single stranded DNA or double stranded DNA) or RNA, or a mix of RNA and DNA nucleotides. In some embodiments, the RNA is one or more selected from a small RNA, ribozyme, small interfering RNA (siRNA), asymmetrical interfering RNA (aiRNA), microRNA (miRNA), Dicer-substrate RNA, double stranded RNA (dsRNA), small hairpin RNA (shRNA), transfer RNA (tRNA), messenger RNA (mRNA), double stranded mRNA, circular RNA (ORNA), guide RNAs, crRNA, tracrRNA, sgRNA, self-amplifying mRNA (SAM), and lentivirus RNA (lgRNA). In some embodiments the polynucleotide is a DNA or RNA selected from aptamers, RNA or DNA containing mobile genetic elements (including, for example, transposons and retrotransposons), and RNA or DNA containing sequences derived from viruses. In some embodiments, the polynucleotide is an antisense oligonucleotide (e.g., from about 8 to about 25 nucleotides), and which may be constructed of DNA, RNA, or a mix of DNA and RNA nucleotides. In various embodiments, the lipid nanoparticles encapsulate a DNA vector, which in some embodiments is a plasmid or linear DNA construct encoding one or more genes under the control of a suitable promoter for delivery. In some embodiments the lipid nanoparticles encapsulate one or more nucleic acid analogs, such as, for example, peptide nucleic acids (PNA) or locked nucleic acids (LNA). In some embodiments the lipid nanoparticles encapsulate one or more Noncoding RNA (including, for example, long noncoding RNA (lncRNA)). In some embodiments the lipid nanoparticles encapsulate one or more polynucleotides containing backbone modifications (including for example phosphothioate bonds), polynucleotides containing one or more base- or sugar-modified nucleosides, polynucleotides chemically conjugated or complexed with proteins or small molecules. In some embodiments the lipid nanoparticles encapsulate one or more coformulations with one or more polynucleotides, one or more proteins and/or one or more small molecules. In some embodiments, the RNA is an mRNA encoding a component of an infectious agent (e.g., an antigen), such as a component of a virus, which is encapsulated with the LNPs to provide for an mRNA vaccine composition. In some embodiments, the LNPs encapsulate at least two or at least three, or at least four open readings frames (as one or more distinct RNA molecules), thereby combining several immunogens for vaccination or proteins for therapy simultaneously.

In other aspects, the present disclosure provides a method for delivering a therapeutic or prophylactic agent, such as a nucleic acid. The method comprises administering to a subject in need thereof the lipid nanoparticle composition of the present disclosure. Exemplary subjects in need of treatment including those needing protection from infectious disease by vaccination, those needing therapy for a genetic disorder, or those needing treatment for cancer. In various embodiments, the compositions are administered by parenteral administration for systemic administration or locally to a target tissue. In various embodiments, the compositions are administered by a route such as intramuscular, intradermal, subcutaneous, intravenous, or intrathecal administration. In other embodiments, the compositions (e.g., including mRNA vaccines) described herein are administered intranasally or by inhalation, or administration to a mucosal surface.

The nanoparticle compositions of this disclosure in some embodiments may be useful for treating a disease, disorder, or condition. In particular, such compositions may be useful in treating a disease, disorder, or condition characterized by missing or aberrant protein or polypeptide activity. For example, a nanoparticle composition comprising an mRNA encoding a missing or aberrant polypeptide may be administered or delivered to a subject. Diseases, disorders, and/or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, and metabolic diseases.

The various aspects and embodiments of this disclosure will be more fully described in connection with the following figures and detailed description.

### BRIEF DESCRIPTION OF THE FIGURES

The foregoing summary, as well as the following detailed description of the invention, will be better understood when read in conjunction with the appended drawings.

FIG. 1 illustrates lipid compounds according to certain embodiments of the disclosure.

FIGS. 2A-C illustrate potency of lipid nanoparticles containing ionizable lipid GILP-124 in HEK293 cells. FIG. 2A shows the structure of the ionizable lipid GILP-124. FIG. 2B shows the results of in vitro evaluation (delivery of luciferase mRNA) of the lipid nanoparticles containing GILP-124 against lipid nanoparticles containing a commercially available ionizable lipid heptadecan-9-yl 8-[2-hydroxyethyl-(6-oxo-6-undecoxyhexyl)amino]octanoate (chemical structure shown in FIG. 7) as a control. FIG. 2C shows the normalized in vitro evaluation.

FIGS. 3A-C illustrate potency of lipid nanoparticles containing the ionizable lipid GILP-124 in HeLa cells. FIG. 3A shows the structure of the ionizable lipid GILP-124. FIG. 3B shows in vitro evaluation (delivery of luciferase mRNA) of lipid nanoparticles containing ionizable lipid GILP-124 against commercially available ionizable lipid heptadecan-9-yl 8-[2-hydroxyethyl-(6-oxo-6-undecoxyhexyl)amino]octanoate as a control. FIG. 3C shows the normalized in vitro evaluation.

FIGS. 4A-C illustrate potency of lipid nanoparticles containing ionizable lipid GILP-126 in HEK293 cells. FIG. 4A shows the structure of the ionizable lipid GILP-126. FIG. 4B shows in vitro evaluation (delivery of luciferase mRNA) of lipid nanoparticles containing ionizable lipid GILP-126 against control ionizable lipid heptadecan-9-yl 8-[2-hydroxyethyl-(6-oxo-6-undecoxyhexyl)amino]octanoate. FIG. 4C shows the normalized in vitro evaluation.

FIGS. 5A-C illustrate potency of lipid nanoparticles containing ionizable lipid GILP-126 in Hela cells. FIG. 5A shows the structure of the ionizable lipid GILP-126. FIG. 5B shows in vitro evaluation (delivery of luciferase mRNA) of lipid nanoparticles containing ionizable lipid GILP-126 against a commercially available ionizable lipid heptadecan-9-yl 8-[2-hydroxyethyl-(6-oxo-6-undecoxyhexyl)amino]octanoate as a control. FIG. 5C shows the normalized in vitro evaluation.

FIG. 6 illustrates endosomal escape ability of LNPs formulated using the ionizable lipids using a hemolysis assay.

FIG. 7 depicts the chemical structure of heptadecan-9-yl 8-[2-hydroxyethyl-(6-oxo-6-undecoxyhexyl)amino]octanoate, which is used as a comparator (control).

FIG. 8A depicts the chemical structure of GILP-133. FIG. 8B depicts the luciferase expression level in HEK293 cells of LNPs comprising GILP-133 in comparison to the control (B).

FIG. 9 depicts the luciferase expression level in Hela cells of LNPs comprising GILP-133 in comparison to the control.

FIG. 10 depicts the luciferase expression level at the injection site (A) and the whole body (B) after the LNPs administered via intramuscular injection to the mice.

FIG. 11 depicts the luciferase expression level in main organs include muscle (A), liver (B), dLN (C), ndLN (D), and spleen (E) 24 hours after administration to the mice.

FIG. 12 depicts IgG levels after injection of LNPs comprising mRNA encoding the SARS-COV-2 Beta spike protein: day 7 (A), day 21 (B), and day 42 (C).

FIG. 13 depicts the number of T cells producing antigen specific IFN-γ in peripheral blood of mice after administration of LNPs comprising mRNA encoding the SARS-COV-2 Beta spike protein: day 7 (A) and day 42 (B).

FIG. 14 illustrates delivery potency of lipid nanoparticles containing GILP-133 (GIL 133) in HEK cells in comparison to MGNR24, MGNR 23, MGNR22, and MGNR18.

FIG. 15 illustrates delivery potency of lipid nanoparticles containing GILP-133 (GLB 133) in HEK293 cells in comparison to BCY-001.

FIG. 16 depicts the cytotoxicity of GILP-133.

FIG. 17 depicts the chemical structure of MGNR23.

### DETAILED DESCRIPTION OF THE INVENTION

The following is a detailed description of the present invention. Those of ordinary skill in the art may make modifications and variations in the embodiments described herein without departing from the spirit or scope of the present disclosure. All publications, patent applications, patents, and other references mentioned herein are expressly incorporated by reference in their entirety.

Unless otherwise defined, all technical and scientific terms used herein have the same meaning as commonly understood by one of ordinary skill in the art to which this invention belongs.

The chemical structures depicted herein are intended to include compounds which differ only in the presence of one or more isotopically enriched atoms. For example, compounds where one or more hydrogen atoms is replaced by deuterium or tritium, or wherein one or more carbon atoms is replaced by 13C- or 14C-enriched carbons, are within the scope of this invention.

The compounds of the present invention may contain asymmetric or chiral centers, and therefore, exist in different stereoisomeric forms. It is contemplated that all stereoisomeric forms of the compound(s) as well as mixtures thereof, including racemic mixtures, form part of the present invention. In addition, the present invention contemplates all geometric and positional isomers. For example, if the compound contains a double bond, both the cis and trans forms (designated as Z and E, respectively), as well as mixtures thereof, are contemplated.

Mixtures of stereoisomers, such as diastereomeric mixtures, can be separated into their individual stereochemical components on the basis of their physical chemical differences by known methods such as chromatography and/or fractional crystallization. Enantiomers can also be separated by converting the enantiomeric mixture into a diastereomeric mixture by reaction with an appropriate optically active compound (e.g., an alcohol), separating the resulting diastereomers and then converting (e.g., hydrolyzing) the individual diastereomers to the corresponding pure enantiomers.

In various aspects and embodiments, the present invention provides lipid nanoparticle compositions comprising an ionizable lipid compound having a plurality of nitrogen atoms in a main chain together with lipophilic substituents as described below. In some embodiments, lipophilic substituents are selected to allow for interactions with encapsulated nucleic acid, such as through cyclic and/or substantially planar moieties. Such ionizable lipid compounds provide advantages for nucleic acid delivery to cells, including but not limited to RNA delivery (e.g., mRNA delivery). As described herein, lipid nanoparticles comprising the ionized lipid described herein have advantages in nucleic acid delivery efficiency, including advantages in endosomal escape.

In various embodiments, the lipid nanoparticle composition comprises an ionizable lipid of Formula (I):

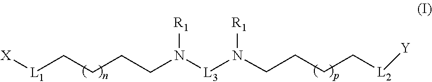

wherein:
each R₁ independently is H or a substituent,
L₁ is —OC(═O)— or —C(═O)O—;
L₂ is —OC(═O) or —C(═O)O—;
L₃ is selected from the group consisting of:

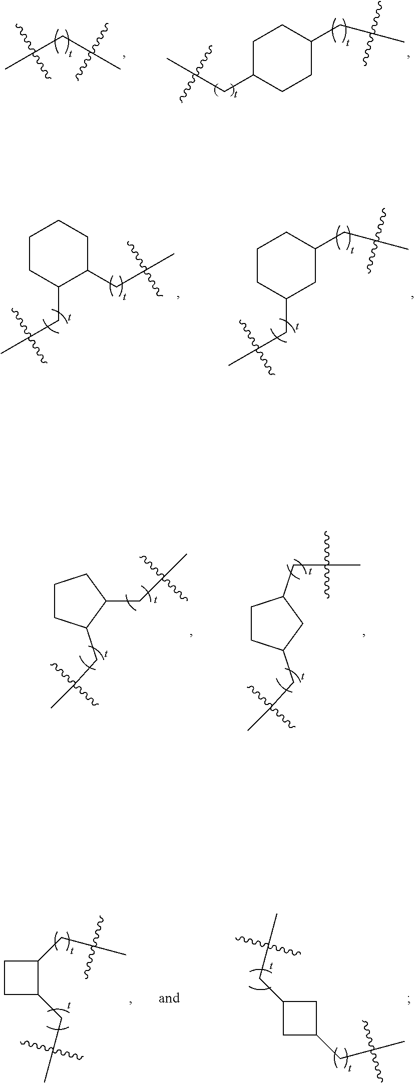

each of n, t, and p is independently selected from 0, 1, 2, 3, 4, 5, 6, 7, 8, 9, and 10;
and X and Y are each independently selected lipophilic moieties having at least six carbon atoms.

In various embodiments, L₃ is selected from the group consisting of:

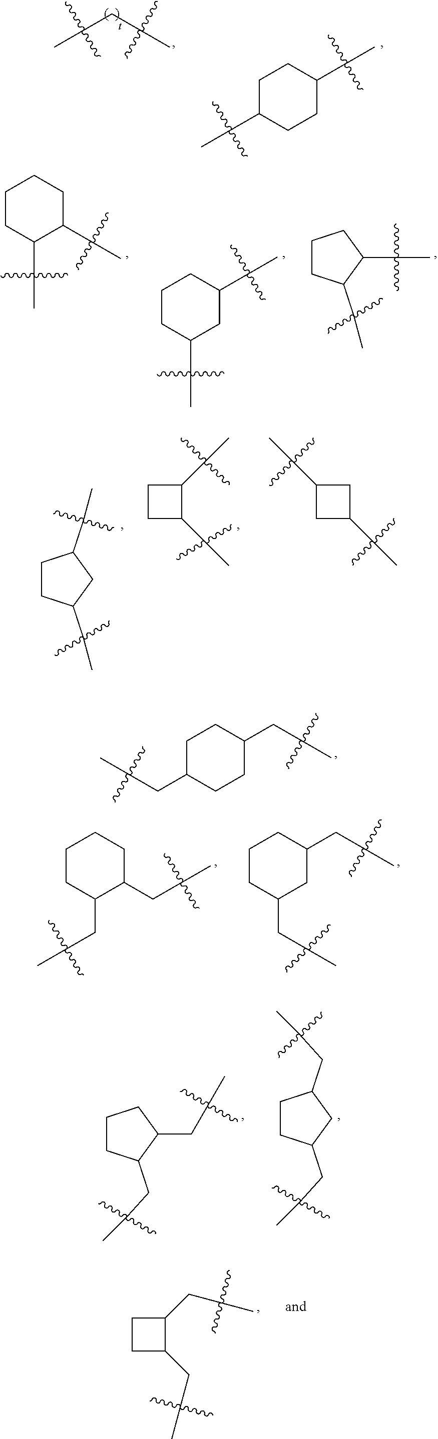

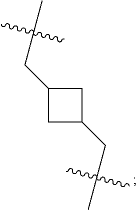

wherein t is selected from 0, 1, 2, 3, 4, 5, 6, 7, 8, 9, and 10.

In various embodiments, L₃ is a bi-cyclic head group selected from the group consisting of 5

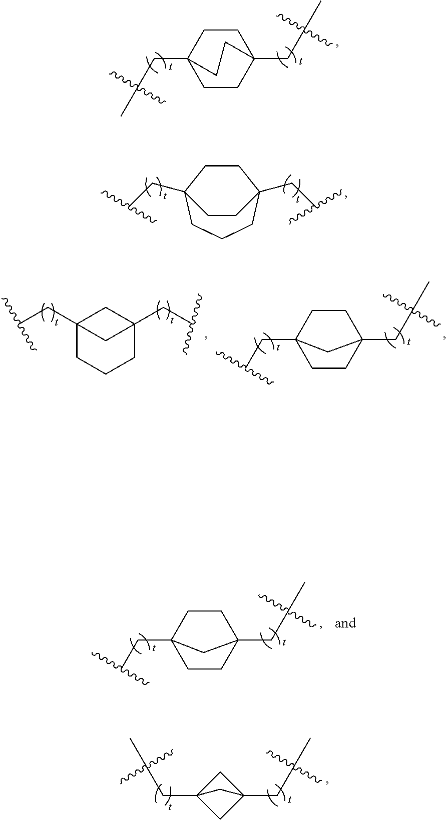

wherein t is 0, 1, 2, 3, 4, 5, 6, 7, 8, 9, and 10; when t is 0, the two N atoms in Formula (I) are connected to the bicycle ring directly.

In various embodiments, one of the two N atoms can be positively charged; or both of the N atoms are positively charged, as shown below. These compounds are within the scope of the present invention.

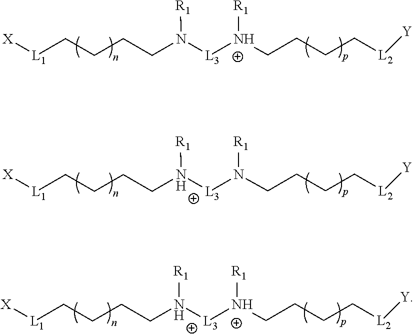

In various embodiments, each R₁ is independently selected from: H or a substituent such as (C₁-C₆)alkyl, substituted (C₁-C₆)alkyl, (C₁-C₆)alkenyl, substituted (C₁-C₆)alkenyl, cycloalkyl, cycloalkenyl, aryl, heteroaryl, and heterocyclo.

In some embodiments, each R₁ is independently selected from (halo)(C₁-C₆)alkyl, (alkoxy)(C₁-C₆)alkyl, (hydroxy)(C₁-C₆)alkyl, —(CH₂)ₙ—S—(C₁-C₆)alkyl, —(CH₂)ₙ—O—(C₁-C₆)alkyl, —C(O)(C₁-C₆)alkyl, (C₃-C₁₂)cycloalkyl, and (C₃-C₁₂)cycloalkenyl, any of which is optionally independently substituted as allowed by valence.

In some embodiments, each R₁ is independently selected from —(CH₂)ₙCHZR′, —CHZR′, —CZ(R′)₂, and —(CH₂)ₙZ, wherein Z is selected from —OC(O)(C₁-C₆)alkyl, —C(O)O(C₁-C₆)alkyl, —OC(O)(C₁-C₆)alkenyl, —C(O)O(C₁-C₆)alkenyl, (C₃-C₁₂)cycloalkyl, and (C₃-C₁₂)cycloalkenyl, any of which is optionally independently substituted as allowed by valence. R′ is a substituent, such as a substituent independently selected from halo, hydroxyl, cyano, nitro, oxo, (C₁-C₆)alkyl, (C₁-C₆)alkoxy, (C₁-C₆) haloalkyl, (C₂-C₆)alkenyl, (C₁-C₆)alkynyl, (C₃-C₁₂)cycloalkyl, (C₃-C₁₂)cycloalkenyl, heterocyclo, aryl, or heteroaryl.

In certain embodiments, each R₁ independently is a C₁ to C₃ alkyl, and which is optionally methyl, ethyl or isopropyl.

In certain embodiments, each R₁ is independently selected from the group consisting of

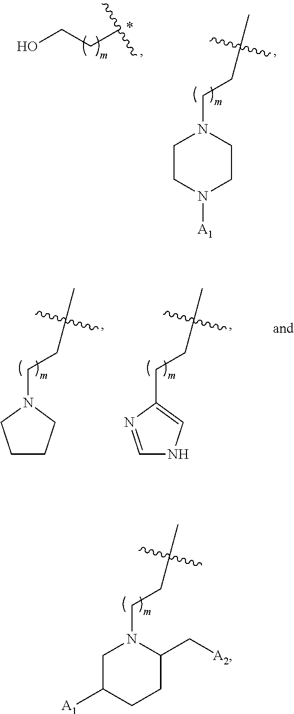

wherein m is 1, 2, 3, 4, 5, 6, 7, 8, 9, or 10; A₁ or A₂ is H, C₁ to C₃ alkyl, and which is optionally methyl, ethyl or isopropyl.

In still other embodiments, each R₁ independently is —(CH₂)ₙZ, and Z is aryl or heterocyclo. For example, each R₁ independently can be —(CH₂)ₙZ, where each Z is selected from phenyl, morpholinyl, pyrrolidinyl, imidazolidinyl, imidazolyl, pyrazolidinyl, pyrazolyl, oxazolidinyl, oxazolyl, pyridinyl, piperidinyl, diazinanyl, and diazinyl.

In exemplary embodiments, each R₁ is independently selected from the group consisting of:

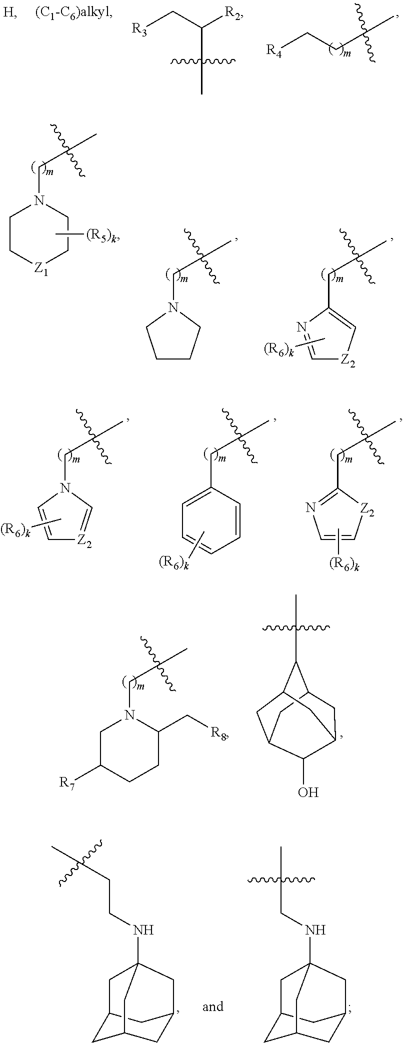

wherein:
each m is independently 0, 1, 2, 3, 4, 5, or 6;
each k is independently 0, 1, 2, 3, or 4;
R₂ is CH₂OH or CO₂ (C₁-C₆)alkyl;
R₃ is OH, (C₁-C₆)alkyl, or aryl;
R₄ is OH, O(C₁-C₆)alkyl, SH, or S(C₁-C₆)alkyl;
each R₅ independently is H, (C₁-C₆)alkyl, —(CH₂) OH, or O(C₁-C₆)alkyl;
each R₆ independently is H, (C₁-C₆)alkyl, —(CH₂) OH, or O(C₁-C₆)alkyl;
R₇ is H or (C₁-C₆)alkyl;\
R₈ is H or (C₁-C₆)alkyl;
Z₁ is NH, O, CH₂, or NRs;
Z₂ is O, S, NR₆, N, or NH.

In various embodiments of Formula I, X and Y are independently selected from linear or branched alkyl, linear or branched alkenyl, sterol, polyphenol, flavonoid, and tocopherol. For example, in some embodiments one or both of X and Y are cholesteryl ester. In some embodiments, one or both of X and Y are ester of tocopherol, which is optionally alpha, beta, gamma, or delta tocopherol. In embodiments, one or both of X and Y are a branched alkyl or alkenyl having from 8 to 30 carbon atoms, and optionally from 10 to 20 carbon atoms, or from 12 to 20 carbon atoms. In some embodiments, one or both of X and Y is/are:

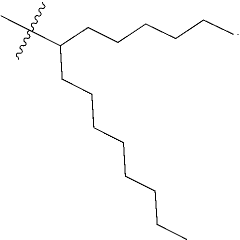 [SMILES: *C(CCCCCC)CCCCCCCC]

In various embodiments, wherein X and/or Y are a flavonoid, the flavonoid is optionally selected from quercetin, rutin, macluraxanthone, genistein, scopoletin, daidzein, taxifolin, naringenin, abyssinones, eriodictyol, fisetin, theaflavin, peonidin, diosmetin, tricin, biochanin, hesperidin, epicatechin, myricetin, kaempferol, luteolin, and apigenin.

In certain embodiments, X is cholesteryl ester and Y is:

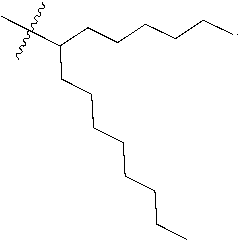 [SMILES: *C(CCCCCC)CCCCCCCC]

In certain embodiments, X is tocopherol, and which is optionally alpha-tocopherol, and Y is:

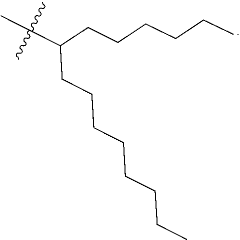 [SMILES: *C(CCCCCC)CCCCCCCC]

In various embodiments of the compound of Formula I, t is an integer from 2 to 10, such as 2, 3, 4, 5, 6, 7, or 8. In some embodiments, t is 3.

In various embodiments of the compounds of Formula I, n and p (which can be the same or different) are each an integer in the range of 3 to 10. In some embodiments, n and p (which can be the same or different) are selected from 6, 7, and 8. In some embodiments, n and p are the same.

In various embodiments, the lipid nanoparticle composition comprises an ionizable lipid of Formula (II), (III) or (IV), wherein n, t, p, L₁, L₂, and R₁ are defined as in Formula (I).

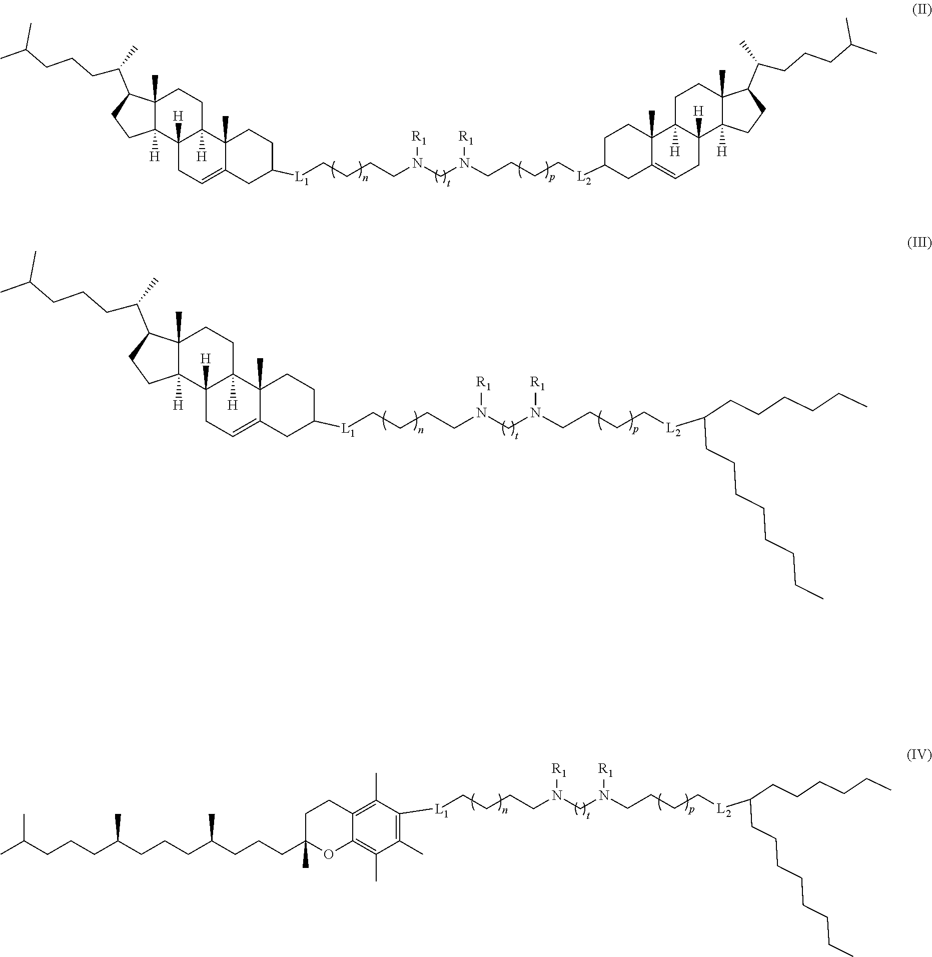

In various embodiments, the composition comprises an ionizable lipid shown in Table 1. Such compounds can be made according to the methods and scheme described in Example 1. Thus, the ionizable lipid of Formula I may be selected from:
(propane-1,3-diylbis(methylazanediyl))bis(heptane-7,1-diyl)bis(2-hexyldecanoate),
(propane-1,3-diylbis(ethylazanediyl))bis(heptane-7,1-diyl)bis(2-hexyldecanoate),
(octane-1,8-diylbis(methylazanediyl))bis(heptane-7,1-diyl)bis(2-hexyldecanoate),
(ethane-1,2-diylbis(benzylazanediyl))bis(heptane-7,1-diyl)bis(2-hexyldecanoate)
(((1R,2R)-cyclohexane-1,2-diyl)bis(methylazanediyl))bis(pentane-5,1-diyl)bis(2-hexyldecanoate),
(propane-1,3-diylbis(isopropylazanediyl))bis(heptane-7,1-diyl)bis(2-hexyldecanoate),
(ethane-1,2-diylbis(tert-butylazanediyl))bis(heptane-7,1-diyl)bis(2-hexyldecanoate),
(bicyclo[1.1.1]pentane-1,3-diylbis(methylazanediyl))bis(heptane-7,1-diyl)bis(2-hexyldecanoate), and
((((1R,3S)-cyclohexane-1,3-diyl)bis(methylene))bis(azanediyl)) bis(heptane-7,1-diyl)bis(2-hexyldecanoate).

In some embodiments, the lipid nanoparticles, e.g., for encapsulating mRNA, may comprise: a cationic or ionizable lipid of Formula I, a neutral lipid, a structural lipid, and a PEGylated lipid. Lipid particle formulations that find use with embodiments of the present disclosure include those described in U.S. Pat. Nos. 8,058,069; 9,738,593; 9,867,888, 10,221,127; 10,166,298; 10,266,485; and 10,442,756, which are hereby incorporated by reference in their entireties. Other lipid nanoparticle formulations known in the art may be employed, including those comprising PLGA or PLA polymers, or poly beta amino ester polymers.

In some embodiments, the lipid nanoparticle (or LNP) comprises a structural lipid. Exemplary structural lipids can be selected from one or more of cholesterol, fecosterol, sitosterol, ergosterol, campesterol, stigmasterol, brassicasterol, tomatidine, ursolic acid, and tocopherols (e.g., alpha tocopherol). In some embodiments, the structural lipid is cholesterol.

In some embodiments, the LNP comprises a one or more phospholipids. Exemplary phospholipids are selected from the group consisting of cardiolipins, sterol modified lipids (modified with a cholesterol moiety attached at the sn-2 carbon of the glycerol backbone), mixed-acyl glycerophospholipids, and symmetrical acyl glycerophospholipids. Head groups for acyl glycerophospholipids include, for example, phosphatidic acid, lysophosphatidic acid, phosphatidylcholine, phosphatidylethanolamine, phosphatidylglycerol, phosphoinositides, and phosphatidylserine. Exemplary phospholipids are selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In various embodiments, the lipid nanoparticle composition further comprises one or more PEG lipids. A PEG lipid is a lipid modified with polyethylene glycol. Exemplary PEG lipids are selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol. A PEG lipid may be selected from PEG-c-DOMG, PEG-DMG, PEG-DLPE, PEG-DMPE, PEG-DPPC, PEG-Cholesterol, PEG tocopherol, or a PEG-DSPE lipid.

In some embodiments, the composition comprises 1,2-dimyristoyl-rac-glycero-3-methoxypolyethylene glycol-2000 (DMG-PEG).

In various embodiments, the lipid nanoparticle composition comprises a structural lipid, a PEG lipid, and a phospholipid, each optionally according to the preceding paragraphs. In exemplary embodiments, the LNP comprises 1,2-distearoyl-sn-glycero-3-phosphocholine (DSPC), cholesterol, and 1,2-dimyristoyl-rac-glycero-3-methoxypolyethylene glycol-2000 (DMG-PEG).

In various embodiments, the molar ratio of the compound of Formula I over the phospholipid in the composition is in the range of about 1:1 to about 10:1: or the range of about 2:1 to about 9:1: or the range of about 3:1 to about 8:1: or the range of about 4:1 to about 7:1; or the range of about 4:1 to about 6:1, and is optionally about 5:1.

In various embodiments, the molar ratio of the phospholipid over the structural lipid in the composition is in the range of about 1:1 to about 1:10; or about 1:2 to about 1:9; or about 1:3 to about 1:8; or about 1:3 to about 1:7; or about 1:3 to about 1:5, and is optionally about 1:4.

In various embodiments, the molar ratio of the structural lipid over the PEG lipid is in the range of about 50:1 to about 1:0.025: or about 40:1 to about 5:1; or about 40:1 to about 10:1: or about 30:1 to about 15:1; or about 30:1 to about 20:1, and is optionally about 50:1.5.

In various embodiments, the molar ratio of the compound of Formula I, the phospholipid, the structural lipid, and the PEG lipid is about 50: about 10: about 38.5: about 1.5 respectively.

A lipid nanoparticle composition may include one or more additional cationic and/or ionizable lipids (i.e., lipids that may have a positive or partial positive charge at physiological pH) in addition to an ionizable lipid according to Formula I. Cationic and/or ionizable lipids may be selected from the following non-limiting group: 3-(didodecylamino)-N1,N1,4-tridodecyl-1-piperazineethanamine (KL10), N1-[2-(didodecylainino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z, 12Z)-octadeca-9,12-dien-1-yloxy]propan-1-amine (Octyl-CLinDMA), (2R)-2-({8-[(3 β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3)-cholest-5-en-3-yloxy]oct 1}oxy)-N,N-dimethyl-3-[(9Z, 12Z)-octadeca-9,12-dien-1-yloxy]propan-1-amine (Octyl-CLinDMA (2S)). In addition to these, a cationic lipid may also be a lipid including a cyclic amine group.

In various embodiments, the largest dimension of the lipid nanoparticles are about 400 nm or less, about 350 nm or less, about 300 nm or less, about 250 nm or less, about 200 nm or less (e.g., about 200 nm or less, about 175 nm or less, about 150 nm or less, about 125 nm, about 100 nm, about 75 nm, about 50 nm or less). Particle size or diameter can be quantified by dynamic light scattering (DLS), transmission electron microscopy, scanning electron microscopy, or another method. In exemplary embodiments, the mean diameter of the lipid nanoparticles is in the range of about 50 nm to about 125 nm (e.g., in the range of about 60 to 110 nm).

In various embodiments, the lipid nanoparticles in the composition encapsulate one or more therapeutic or diagnostic agents.

For example, the lipid nanoparticles can encapsulate one or more polynucleotide, which can be DNA (single stranded DNA or double stranded DNA) or RNA, or a mix of RNA and DNA nucleotides. In some embodiments, the RNA is one or more selected from a small RNA, ribozyme, small interfering RNA (siRNA), asymmetrical interfering RNA (aiRNA), microRNA (miRNA), Dicer-substrate RNA, double-stranded RNA (dsRNA), small hairpin RNA (shRNA), transfer RNA (tRNA), messenger RNA (mRNA), double stranded mRNA, circular RNA (ORNA). and self-amplifying mRNA (SAM). In some embodiments, the polynucleotide is an antisense oligonucleotide (e.g., from about 8 to about 25 nucleotides), and which may be constructed of DNA, RNA, or a mix of DNA and RNA nucleotides. Antisense oligonucleotides may include various sugar modifications known in the art (e.g., LNA, CET, 2′-MOE, 2′-OMe, 2′-F) as well as various known backbone modifications (e.g., phosphorothioate, morpholino, PNA).

In various embodiments, the lipid nanoparticles encapsulate mRNA. mRNA useful in the disclosure typically include a first region of linked nucleosides encoding a polypeptide of interest (e.g., a coding region), a first flanking region located at the 5′-terminus of the first region (e.g., a 5′-UTR), a second flanking region located at the 3′-terminus of the first region (e.g., a 3′-UTR). The mRNA may comprise a 5′-cap region and a 3′-stabilizing region (e.g., a poly-A tail). In some embodiments, a nucleic acid or polynucleotide includes a Kozak sequence (e.g., in the 5′-UTR). In some cases, mRNA may contain one or more intronic nucleotide sequences capable of being excised from the polynucleotide.

Nucleic acids and polynucleotides may include naturally occurring “canonical” nucleotides A (adenosine), G (guanosine), C(cytosine), U (uridine), and T (thymidine). Nucleic acids and polynucleotides may further include one or more non-canonical nucleotides such as 1-methylpseudouridine (mlΨ′) and pseudouridine (Y), in place of some or all uridines in an RNA. Modified nucleosides are described in U.S. Pat. No. 8,691,966 and WO2013022990A1, which are incorporated by reference in their entirety.

In accordance with this disclosure, the mRNA comprises modified uridines. In some embodiments, the modified uridines are selected from pseudouridine (Y), N1-methylpseudouridine and 5-methoxy-uridine. For example, at least about 10%, or at least about 25%, or at least about 50%, or at least about 75%, or all uridines can be modified uridines, such as pseudouridine, N1-methylpseudouridine, and/or 5-methoxy-uridine. In some embodiments, substantially all uridines of the mRNA are replaced with pseudouridine and/or N1-methylpseudouridine.

In various embodiments, the nucleic acid (e.g., RNA or mRNA) further includes one or more modified nucleotides selected from: 2-thiouridine, 5-azauridine, 4-thiouridine, 5-methyluridine, 5-methylpseudouridine, 5-aminouridine, 5-aminopseudouridine, 5-hydroxyuridine, 5-hydroxy pseudouridine, 5-methoxypseudouridine, 5-ethoxyuridine, 5-ethoxypseudouridine, 5-hydroxy methyluridine, 5-ydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxy pseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine. N4-aminocytidine, N4-hydroxycytidine, 5-methylcytidine, 5-aminocytidine, 5-hydroxycytidine, 5-methoxycytidine, 5-ethoxy cytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine. N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine. 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine. N4-methyl-5-methyl-5-azacytidine, N4-methyl-5-amino-5-azacytidine, N4-methyl-5-hydroxy-5-azacytidine. N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine. N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine. 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine. 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-2-thio-N4-hydroxy-5-hydroxypseudoisocytidine, N6-aminopseudoisocytidine, methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosin.

In various embodiments, the lipid nanoparticles encapsulate a DNA vector, which in some embodiments is a plasmid or linear DNA construct encoding one or more genes under the control of a suitable promoter for delivery.

In some cases, the polynucleotide is greater than 10 nucleotides in length (e.g., an oligonucleotide). In various embodiment, the polynucleotide is at least 25, or at least about 50 nucleotides, or at least about 100 nucleotides in length (e.g., an small RNA or siRNA). In various embodiments, the length is at least 100 nucleotides. In some embodiments, the length of the polynucleotide (e.g., mRNA or encoding DNA) is at least about 200 nucleotides, or at least about 300 nucleotides, or at least about 500 nucleotides in length, or at least about 700 nucleotides in length, or at least about 1000 nucleotides, or at least about 1200 nucleotides in length, or at least about 1500 nucleotides in length, or at least 2000 nucleotides in length, or at least about 3000 nucleotides, or at least about 4000 nucleotides, or at least about 5000 nucleotides, or at least about 6000 nucleotides, or at least about 7000 nucleotides, or at least about 8000 nucleotides, or at least about 9000 nucleotides, or at least about 10000 nucleotides.

In some embodiments, the RNA is an mRNA encoding a component of an infectious agent, such as a component of a virus, which is encapsulated with the LNPs to provide for an mRNA vaccine composition. In some embodiments, the LNPs encapsulate at least two or at least three, or at least four open readings frames, thereby combining several immunogens for vaccination or proteins for therapy simultaneously.

In some embodiments the RNA is an RNA described in WO2022/016077, US 2022/0370599, or WO2021/113774.

In some embodiments, the mRNA encodes one or more proteins of a virus or one or more polypeptides derived from virus proteins, for example, a DNA or RNA virus. Examples include those of the family Paramyxoviridae and/or genus Pneumovirinae or Morbillivirus. Example viruses include human metapneumovirus (hMPV), parainfluenza virus (hPIV), (types 1, 2, and 3), respiratory syncytial virus (RSV), and Measles virus (MeV). In some embodiments, the RNA virus is a coronavirus (CoV)(subfamily Coronavirinae, of the family Coronaviridae). In some embodiments, the coronavirus is a betacoronavirus, such as SARS-COV or MERS-COV. In some embodiments, the RNA virus is SARS-COV-2, or a natural variant thereof. In other embodiments, the virus is a herpes virus, such as a herpes simplex virus or varicella zoster virus. In other embodiments, the virus is RSV, a hepatitis virus, or an adenovirus. In still other embodiments, the virus is an Ebola virus.

In some embodiments, the mRNA encodes one or more viral structural proteins or one or more polypeptides derived from virus proteins, such as a protein comprised in the viral envelop, such as a Spike protein(S) for coronaviruses. Alternatively or in addition, the mRNA encodes other CoV structural proteins such as M (membrane)glycoprotein, E (envelope) protein, and/or N (nucleocapsid) protein. Alternatively, an mRNA encoding the Spike protein or other structural protein can be encapsulated in particles that comprise or are decorated with one or more CoV structural proteins or portions thereof.

In some embodiments, the mRNA encodes one or more influenza proteins, such as neuraminidase (NA), hemagglutinin (HA), matrix protein 2 (M2), and/or nucleoprotein (NP). In some embodiments the mRNA encodes at least one neuraminidase and at least one hemagglutinin.

In some embodiments the mRNA encodes one or more varicella antigens, such as glycoprotein E, glycoprotein B, glycoprotein H, glycoprotein L, or glycoprotein I.

In some embodiments the mRNA encodes one or more cancer-associated epitopes or neoantigens.

In some embodiments, mRNA is targeted for expression in tissue or organs selected from liver (e.g., hepatocytes), skin (e.g., keratinocytes), skeletal muscle, endothelial cells, epithelial cells of various organs including the lungs, or hematopoietic or immune cells (e.g., T cells, B cells, or macrophages), for example. For example, the mRNA may be designed to encode polypeptides of interest selected from vaccine targets, enzymes (including metabolic enzymes or endonucleases such as Cas endonucleases), antibodies or antigen-binding fragments thereof or antibody mimetics (including nanobodies or single chain antibodies such as single chain variable fragments), secreted proteins or peptides (including cytokines, growth factors, or soluble receptors for the same), plasma membrane proteins, cytoplasmic or cytoskeletal proteins, intracellular membrane bound proteins, nuclear proteins, proteins associated with human disease (including proteins having loss-of-function or gain-of-function mutations associated with human disease). In some embodiments, the therapeutic protein includes one or more cancer-associated epitopes (e.g., one or more mutations associated with cancer, including neoantigens), which may find use in a cancer vaccine. An exemplary embodiment in which the mRNA encodes for an antibody, open reading frames encoding heavy and light chains can be expressed from different mRNA molecules.

In various embodiments, the nucleic acid encodes a therapeutic protein, e.g., for treatment of a disease or disorder. Exemplary diseases characterized by dysfunctional or aberrant protein activity include cystic fibrosis, sickle cell anemia, epidermolysis bullosa, amyotrophic lateral sclerosis, and glucose-6-phosphate dehydrogenase deficiency. In various embodiments, the nucleic acid (e.g., mRNA) encodes a protein that overcomes an aberrant protein activity present in the cell of a subject. Specific examples of a dysfunctional protein are the missense mutation variants of the cystic fibrosis transmembrane conductance regulator (CFTR) gene, which produce a dysfunctional protein variant of CFTR protein, which causes cystic fibrosis. Other diseases characterized by missing or substantially diminished protein activity (such that proper, normal or physiological protein function does not occur) include cystic fibrosis, Niemann-Pick type C, β thalassemia major, Duchenne muscular dystrophy, Hurler Syndrome, Hunter Syndrome, and Hemophilia A. In these conditions, proteins important for cellular function may not be present, or are essentially non-functional. The present invention provides nanoparticle compositions for treating such conditions or diseases. The nanoparticles encapsulate nucleic acids, such as mRNA, that encodes for a protein that replaces the protein activity missing from the target.

In some embodiments in which the composition is a vaccine, the composition may comprise one or more adjuvants. Such adjuvants may include, for example, MIPLA, R848, QS-21, aluminum salt-based adjuvants: emulsion adjuvants, such as MF59 (squalene, polysorbate 80, sorbitan trioleate, trisodium citrate dehydrate) and AS03 (polysorbate 80, squalene, DL-α-tocopherol): TLR agonist based adjuvants, such as CpG ODN and AS04 (3′-O-deacylated monophosphoryl lipid A (MPL)+aluminum salt): AS01B (MPL+QS-21); and any analogues of the foregoing. Other adjuvants of use with the present invention may include Glucopyranosyl Lipid Adjuvant (GLA), CpG oligodeoxynucleotides (e.g., Class A or B), poly(I: C), aluminum hydroxide, Pam3CSK4; and any analogues of the foregoing. Other adjuvants of use with the present invention may include lipid based adjuvants, such as GLA-SE and GLA-AF: emulsions, such as Montanide ISA 51 and Montanide ISA 720: Saponins such as Matrix M and ASO2; nucleotides such as cyclic dinucleotides (CDNs), CpG, ODN, dsRNA, IL-12, and Pika adjuvants: cytokines, such as IL-2, IL-12, IL-15, and granulocyte-macrophage-colony-stimulating factor (GM-CSF): calcium phosphate: bacterial flagellin: virosomes; and any analogues of the foregoing

In some embodiments, for the encapsulation of nucleic acid, the ratio between the amines of the ionizable lipid of Formula I and phosphates of the polynucleotide (N: P ratio) is from about 2:1 to about 50:1, or about 2:1 to about 40:1, or about 2:1 to about 20:1 or about 2:1 to about 15:1, or about 2:1 to about 12:1, or about 35:1 to about 45:1, or about 2:1 to about 10:1, or about 3:1 to about 10:1, or about 4:1 to about 10:1, or about 5:1 to about 7:1, or about 35:1 to about 45:1. In some embodiments, the N: P ratio is about 6:1. In some embodiments the N: P ration is about 12:1.

In some embodiments, the lipid nanoparticles of this disclosure encapsulate RNA. The RNA in various embodiments is included in the composition at a concentration of from about 0.01 to about 2.0 mg/mL, or from about 0.01 to about 1.0 mg/mL, or from about 0.05 to about 0.5 mg/mL, or about 0.1 mg/mL.

In addition to nucleic acids, in other embodiments the therapeutic can be another biologically active substance or “active agent.” A therapeutic and/or prophylactic may be a substance that, once delivered to a cell or organ, brings about a desirable change in the cell, organ, or other bodily tissue or system. In some embodiments, a therapeutic and/or prophylactic is a small molecule drug useful in the treatment of a particular disease, disorder, or condition. Examples of drugs useful in the nanoparticle compositions include, but are not limited to, antineoplastic agents (e.g., vincristine, doxorubicin, mitoxantrone, camptothecin, cisplatin, bleomycin, cyclophosphamide, methotrexate, and streptozotocin), antitumor agents (e.g., actinomycin D, vincristine, vinblastine, cystine arabinoside, anthracyclines, alkylative agents, platinum compounds, antimetabolites, and nucleoside analogs, such as methotrexate and purine and pyrimidine analogs), anti-infective agents, local anesthetics (e.g., dibucaine and chlorpromazine), beta-adrenergic blockers (e.g., propranolol, timolol, and labetolol), antihypertensive agents (e.g., clonidine and hydralazine), anti-depressants (e.g., imipramine, amitriptyline, and doxepim), anti-conversants (e.g., phenytoin), antihistamines (e.g., diphenhydramine, chlorphenirimine, and promethazine), antibiotic/antibacterial agents (e.g., gentamycin, ciprofloxacin, and cefoxitin), antifungal agents (e.g., miconazole, terconazole, econazole, isoconazole, butaconazole, clotrimazole, itraconazole, nystatin, naftifine, and amphotericin B), antiparasitic agents, hormones, hormone antagonists, immunomodulators, neurotransmitter antagonists, antiglaucoma agents, vitamins, narcotics, and imaging agents.

In some embodiments, a therapeutic and/or prophylactic is a cytotoxin, a radioactive ion, a chemotherapeutic, a vaccine, a compound that elicits an immune response. A cytotoxin or cytotoxic agent includes any agent that may be detrimental to target cells.

The nanoparticle compositions described herein are stable for storage and/or shipment when refrigerated or frozen (e.g., being stored at a temperature of 4° C. or lower, such as a temperature between about −150° C. and about 0° C. or between about −80° C. and about −20° C. In some embodiments, the pharmaceutical composition is stable when refrigerated for storage and/or shipment at, for example, about 0° C., or about −10° C., or about −20° C., or about −30° C., or about −40° C., or −50° C., or −60° C., or −70° C., or about −80° C.

In certain embodiments, the composition is stable at refrigerated temperatures. For example, in some embodiments, the lipid nanoparticles are stable for at least three months at 2° C., or are stable for at least six months at 2° C. In some embodiments, the composition is stable for at least three months at 4° C., or is stable for at least six months at 4° C. In some embodiments, the composition is stable for at least three months at 8° C., or is stable for at least six months at 8° C. The compositions can therefore be stored and/or distributed at temperatures in the range of 2-8° C., providing substantial advantages over currently authorized mRNA vaccines.

Degradation or instability can be determined by an increase or decrease in average size of the particles in the formulation (e.g., an average size that is at least about 10% or at least about 20% larger or smaller than controls).

RNA degradation can be determined by the presence of smaller RNA species and disappearance of the desired RNA size, as determined for example by high performance liquid chromatography (HPLC).

In various embodiments, the population of LNP encapsulating the RNA is relatively homogenous, as determined by a polydispersity index (PDI), which indicates the particle size distribution of the lipid nanoparticles. A small (e.g., less than 0.3) PDI generally indicates a narrow particle size distribution. A LNP may have a PDI from about 0 to about 0.25. In some embodiments, the PDI is from about 0.10 to about 0.20.

In various embodiments, the compositions have relatively low charges, positive or negative, as more highly charged species may interact undesirably with cells or tissues in the body upon administration. In some embodiments, the zeta potential of a composition may be from about-20 mV to about +20 mV, or from about-10 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least about 50%, or at least about 70%, or at least about 80%, or at least about 90%.

A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements. In various embodiments, the composition comprises excipients which can include one or more antioxidants, non-ionic surfactants, one or more stabilizing agents, and a pH buffer.

Exemplary antioxidants include methionine, propyl gallate, ascorbic acid, citric acid, monothioglycerol, phosphoric acid, potassium metabisulfite, alpha-tocopherol, sodium sulfite, cysteine, sodium metabisulfite, t-cysteine-HCL, vitamin E TPGS, HP-β-CD, Butylhydroxyanisol (BHA), Butylated hydroxytoluene (BHT), L-methionine, L-cysteine, citric acid/citrate, vitamin A, vitamin C(ascorbic acid), vitamin E, or other antioxidant approved for use injectable use in humans. Further exemplary antioxidants, such as polyphenols or vitamin P, not yet approved for injectable use may also be used. In various embodiments the concentration of the antioxidant is from 0.01% to about 1.50% w/v, or from about 0.02% to about 0.2%, or from about 0.05% to about 0.5% or from about 0.10% to about 0.25% w/v.

In various embodiments, the composition comprises a non-ionic surfactant, such as a polysorbate or a poloxamer. In some embodiments, the polysorbate is polysorbate-20, polysorbate-40, polysorbate-60, and/or polysorbate-80. In some embodiments, the non-ionic surfactant is polysorbate-20. In various embodiments, the concentration of polysorbate-20 is from about 0.001% to about 0.1% w/v, or from about 0.005% to about 0.05% w/v, or about 0.01% w/v. In some embodiments the poloxamer is Poloxamer 188, Poloxamer 124, Poloxamer 182, Poloxamer 331, Poloxamer 335, Poloxamer 407, or other Poloxamers.

In various embodiments, the stabilizing agent is selected from one or more of glycine, sorbitol, and gelatin. In some embodiments, the stabilizing agent is glycine, which can be present in the composition at a concentration of from about 0.25% to about 15% w/v, or from about 0.25% to about 10% w/v, or from about 0.25% to about 5% w/v, or from about 0.5% to about 2.5% w/v. In some embodiments, the concentration of glycine is about 1.5% w/v. In these or other embodiments the stabilizing agent is sorbitol, which is optionally present in the composition at from about 1% to about 20%, such as about 10% w/v. In these or other embodiments the stabilizing agent(s) comprise gelatin, which is optionally present in the composition at from about 1% to about 20% w/v, or from about 5% to about 15% w/v, such as about 10% w/v.

In various embodiments the pH is buffered at a pH from about 6.0 to about 8.0. In certain embodiments the pH is buffered at about 6.0, about 7.4, or at about 8.0. In various embodiments, the composition is pH buffered at about pH 7.4. In various embodiments, the pH buffer is a phosphate buffer. In still other embodiments, the pH buffer is a Tris-EDTA (TE) buffer. In some embodiments the pH buffer is a histidine buffer. In some embodiments, the histidine buffer is L-Histidine. In some embodiments the buffer is a TE buffer, consisting of tris HCL and disodium EDTA. In some embodiments the buffer is tris acetate, which can consist of tris base and sodium acetate. In some embodiments, the buffer is sodium citrate buffer, which can consist of sodium citrate dihydrate and citric acid. In some embodiments, the buffer is PBS, which can consist of potassium chloride, monobasic potassium phosphate, sodium chloride, and dibasic sodium phosphate dihydrate.

In some embodiments, the composition further comprises a metal ion chelator. For example, the chelator may be selected from ethylenediaminetetraacetic acid (EDTA), diethylenetriamine pentaacetic acid (DTPA), ethylene glycol-bis(β-aminoethyl ether)-N,N,N′,N′-tetraacetic acid (EGTA), iminodisuccinic acid, polyaspartic acid, ethylenediamine-N,N′-disuccinic acid (EDDS), methylglycine diacetic acid (MGDA), L-glutamic acid N,N-diacetic acid (GLDA), or a salt thereof. In some embodiments, the metal ion chelator is EDTA or salt thereof, which is optionally disodium EDTA. In some embodiments, the concentration or EDTA or disodium EDTA is from about 0.01 mM to about 1 mM, or from about 0.05 mM to about 0.5 mM, or about 0.1 mM.

In various embodiments, the composition further comprises an excipient that reduces exposure of the RNA to water. In some embodiments, the excipient that reduces exposure of the mRNA to water is a saccharide, such as sucrose.

In some embodiments, the composition further comprises an excipient that reduces degradation of the RNA by free-radical oxidation. In some embodiments, the excipient that reduces degradation of the RNA by free-radical oxidation is one or more of ethanol and histidine. In some embodiments, ethanol is included as an excipient at 200 mM of less, or about 150 mM or less, or about 100 mM of less, or about 50 mM or less, to avoid effects on LNP size. In some embodiments, the excipient(s) that reduce degradation of the RNA comprise or consist of histidine. Histidine may be present in the composition at a concentration of from about 0.01% w/v to about 1% w/v, or from about 0.05% w/v to about 0.5% w/v, or about 0.1% w/v.

In other aspects, the present disclosure provides a method for delivering a therapeutic agent. The method comprises administering to a subject in need thereof the lipid nanoparticle composition of the present disclosure. Exemplary subjects and conditions or disorders in need of treatment (including protection from infectious disease by vaccination) are already described.

In some aspects, the disclosure provides a method for preventing or reducing the probability of a viral infection in a patient or a population, such as SARS-COV-2 infection. In these embodiments, the method comprises administering an mRNA vaccine of the present disclosure expressing one or more viral proteins, such as SARS-COV-2 Spike protein and/or other SARS-COV-2 structural protein as described herein. In some embodiments, the mRNA vaccine is administered as a single dose. In some embodiments, the mRNA vaccine is administered as multiple (e.g. two or three) doses, with a booster one, two, or three weeks after an initial dose. Periodic boosters can be administered as needed. In accordance with the various aspects, the present disclosure provides for simplified global distribution over currently available mRNA vaccines, since sub-zero conditions are not required for storage and distribution and/or because stability of the vaccine is improved.

In some embodiments of this aspect, the disclosure provides a method for expressing a therapeutic protein in a patient, comprising administering the mRNA composition described herein. For example, diseases, disorders, and/or conditions for treatment or prevention, include: autoimmune disorders (e.g., diabetes, lupus, multiple sclerosis, psoriasis, rheumatoid arthritis): inflammatory disorders (e.g., arthritis, pelvic inflammatory disease): infectious diseases (e.g., viral infections, bacterial infections, fungal infections, and sepsis): neurological disorders (e.g., Alzheimer's disease, Huntington's disease: autism: Duchenne muscular dystrophy): cardiovascular disorders (e.g., atherosclerosis, hypercholesterolemia, thrombosis, clotting disorders, angiogenic disorders such as macular degeneration); metabolic disorders and liver disorders (e.g., ornithine transcarbamylase deficiency): proliferative disorders (e.g., cancer, benign neoplasms): respiratory disorders (e.g., chronic obstructive pulmonary disease or idiopathic pulmonary fibrosis): digestive disorders (e.g., inflammatory bowel disease, ulcers): musculoskeletal disorders (e.g., fibromyalgia, arthritis); endocrine, metabolic, and nutritional disorders (e.g., diabetes, osteoporosis): urological disorders (e.g., renal disease): psychological disorders (e.g., depression, schizophrenia): skin disorders (e.g., wounds, eczema); and blood and lymphatic disorders (e.g., anemia, hemophilia).

In some embodiments, the therapeutic agent (such as an RNA) of a pharmaceutical compositions in accordance with the present disclosure may be administered at a dose of about 1 μg to 500 μg, or about 5 μg to 450 μg, or about 10 μg to 400 μg, or about 15 μg to 400 μg, or about 20 μg to 350 μg, or about 25 μg to 325 μg, or about 30 μg to 300 μg, or about 35 μg to 275 μg, or about 40 μg to 250 μg, or about 45 μg to 225 μg, or about 50 μg to 200 μg, or about 60 μg to 180 μg, or about 70 μg to 150 μg, or about 80 μg to 125 μg, or about 90 μg to 100 μg. In some embodiments, the therapeutic agent is mRNA vaccine.

In various embodiments, the subject is an mammal or a bird. In some embodiments, the subject is a human. Other exemplary subjects include pigs, dogs, cats, cows, horses, sheep, and chickens.

In various embodiments, the compositions are administered by parenteral administration for systemic administration or locally to a target tissue. In various embodiments, the compositions are administered by a route such as intramuscular, intradermal, subcutaneous, intravenous, or intrathecal administration. In other embodiments, the compositions (e.g., mRNA vaccines) described herein are administered intranasally or by inhalation.

In various embodiments, a nanoparticle composition of the disclosure may target or accumulate in a particular type or class of cells or tissues, such as liver, kidney, spleen, lung, heart, muscle, or CNS. Specific delivery to a particular class of cells, an organ, or a system or group thereof implies that a higher proportion of nanoparticle are delivered to the destination (e.g., tissue) of interest relative to other destinations, e.g., upon administration of a nanoparticle composition to a mammal. In some embodiments, specific delivery may result in a greater than 2 fold, 5 fold, 10 fold, 15 fold, or 20 fold increase in the amount of therapeutic and/or prophylactic per 1 g of tissue of the targeted destination (e.g., tissue of interest, such as a liver) as compared to another destination (e.g., the spleen). In some embodiments, the target tissue is a tumor.

The nanoparticle compositions of this disclosure in some embodiments may be useful for treating a disease, disorder, or condition. In particular, such compositions may be useful in treating a disease, disorder, or condition characterized by missing or aberrant protein or polypeptide activity. For example, a nanoparticle composition comprising an mRNA encoding a missing or aberrant polypeptide may be administered or delivered to a cell. Subsequent translation of the mRNA may produce the polypeptide, thereby reducing or eliminating an issue caused by the absence of or aberrant activity caused by the polypeptide. Diseases, disorders, and/or conditions characterized by dysfunctional or aberrant protein or polypeptide activity for which a composition may be administered include, but are not limited to, rare diseases, infectious diseases (as both vaccines and therapeutics), cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis.

### Definitions

The articles “a” and “an” as used herein and in the appended claims are used herein to refer to one or to more than one (i.e., to at least one) of the grammatical object of the article unless the context clearly indicates otherwise. By way of example, “an element” means one element or more than one element.

The present disclosure contemplates the inclusion of one or more substituents of the ionizable lipid of Formula I. A group or atom that replaces a hydrogen atom is also called a “substituent.” In various embodiments, a particular molecule or group can have one or more substituent depending on the number of hydrogen atoms that can be replaced. The term “H” denotes a single hydrogen atom, and is not a substituent.

Where the term “alkyl” is used, either alone or within other terms such as “haloalkyl” or “alkylamino”, embraces linear or branched hydrocarbon radicals. Exemplary alkyls have from one to about thirty carbon atoms. Examples of alkyls include methyl, ethyl, n-propyl, isopropyl, n-butyl, isobutyl, sec-butyl, tert-butyl, pentyl, isoamyl, hexyl and the like. The term “alkylenyl” or “alkylene” embraces bridging divalent alkyl radicals such as methylenyl or ethylenyl.

The term “alkenyl” embraces linear or branched hydrocarbon radicals having at least one carbon-carbon double bond. Exemplary alkenyl groups have from two to about thirty carbon atoms. Examples of alkenyl radicals include ethenyl, propenyl, allyl, propenyl, butenyl and 4-methylbutenyl. The term “alkenyl” embraces radicals having “cis” and “trans” orientations, or alternatively, “E” and “Z” orientations.

The term “alkynyl” denotes linear or branched radicals having at least one carbon-carbon triple bond. Exemplary alkynyl groups have two to about thirty carbon atoms. Examples of such radicals include propargyl, and butynyl, and the like.

Alkyl, alkylenyl, alkenyl, and alkynyl radicals may be optionally substituted with one or more functional groups such as halo, hydroxy, nitro, amino, cyano, haloalkyl, aryl, heteroaryl, and heterocyclo and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for example, may have either an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals, e.g., having one to about thirty carbon atoms any one of which may be substituted with one or more hydroxyl radicals. Examples of such radicals include hydroxymethyl, hydroxyethyl, hydroxypropyl, hydroxy butyl and hydroxyhexyl.

The term “alkoxy” embraces linear or branched oxy-containing radicals each having alkyl portions, e.g., of one to about thirty carbon atoms. Examples of such radicals include methoxy, ethoxy, propoxy, butoxy and tert-butoxy. Alkoxy radicals may be further substituted with one or more halo atoms, such as fluoro, chloro or bromo, to provide “haloalkoxy” radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one or more rings, wherein such rings may be attached together in a fused manner. The term “aryl” embraces aromatic radicals such as phenyl, naphthyl, indenyl, tetrahydronaphthyl, and indanyl. An “aryl” group may have 1 or more substituents such as lower alkyl, hydroxyl, halo, haloalkyl, nitro, cyano, alkoxy, and lower alkylamino, and the like.

The term “heterocyclyl” (or “heterocyclo”) embraces saturated, partially saturated and unsaturated heteroatom-containing ring radicals, where the heteroatoms may be selected from nitrogen, sulfur and oxygen. It does not include rings containing —O—O—, —O—S— or —S—S— portions. The “heterocyclyl” group may have 1 to 4 substituents such as hydroxyl, Boc, halo, haloalkyl, cyano, lower alkyl, lower aralkyl, oxo, lower alkoxy, amino and lower alkylamino.

Examples of saturated heterocyclic radicals include saturated 3 to 6-membered heteromonocyclic groups containing 1 to 4 nitrogen atoms[e.g., pyrrolidinyl, imidazolidinyl, piperidinyl, pyrrolinyl, piperazinyl]: saturated 3 to 6-membered heteromonocyclic group containing 1 to 2 oxygen atoms and 1 to 3 nitrogen atoms[e.g., morpholinyl]: saturated 3 to 6-membered heteromonocyclic group containing 1 to 2 sulfur atoms and 1 to 3 nitrogen atoms[e.g., thiazolidinyl]. Examples of partially saturated heterocyclyl radicals include dihydrothienyl, dihydropyranyl, dihydrofuryl and dihydrothiazolyl.

Examples of unsaturated heterocyclic radicals, also termed “heteroaryl” radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl[e.g., 4H-1,2,4-triazolyl, 1H-1,2,3-triazolyl, 2H-1,2,3-triazolyl]: unsaturated 5- to 6-membered heteromonocyclic group containing an oxygen atom, for example, pyranyl, 2-furyl, 3-furyl, etc.: unsaturated 5 to 6-membered heteromonocyclic group containing a sulfur atom, for example, 2-thienyl, 3-thienyl, etc.: unsaturated 5- to 6-membered heteromonocyclic group containing 1 to 2 oxygen atoms and 1 to 3 nitrogen atoms, for example, oxazolyl, isoxazolyl, oxadiazolyl[e.g., 1,2,4-oxadiazolyl, 1,3,4-oxadiazolyl, 1,2,5-oxadiazolyl]: unsaturated 5 to 6-membered heteromonocyclic group containing 1 to 2 sulfur atoms and 1 to 3 nitrogen atoms, for example, thiazolyl, thiadiazolyl[e.g., 1,2,4-thiadiazolyl, 1,3,4-thiadiazolyl, 1,2,5-thiadiazolyl].

The term heterocyclyl, (or heterocyclo) also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl[e.g., tetrazolo[1,5-b]pyridazinyl]: unsaturated condensed heterocyclic group containing 1 to 2 oxygen atoms and 1 to 3 nitrogen atoms[e.g. benzoxazolyl, benzoxadiazolyl]: unsaturated condensed heterocyclic group containing 1 to 2 sulfur atoms and 1 to 3 nitrogen atoms[e.g., benzothiazolyl, benzothiadiazolyl]; and saturated, partially unsaturated and unsaturated condensed heterocyclic group containing 1 to 2 oxygen or sulfur atoms[e.g. benzofuryl, benzothienyl, 2,3-dihydro-benzo[1,4]dioxinyl and dihydrobenzofuryl]. Examples of heteroaryl radicals include quinolyl, isoquinolyl, imidazolyl, pyridyl, thienyl, thiazolyl, oxazolyl, furyl and pyrazinyl. Other heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

Particular examples of non-nitrogen containing heteroaryl include pyranyl, 2-furyl, 3-furyl, 2-thienyl, 3-thienyl, benzofuryl, and benzothienyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1\′-benzo[d]isothiazol-6-yl, dihydropyranyl, dihydrofuryl and dihydrothiazolyl, and the like.

The term “heterocyclo” thus encompasses the following ring systems:

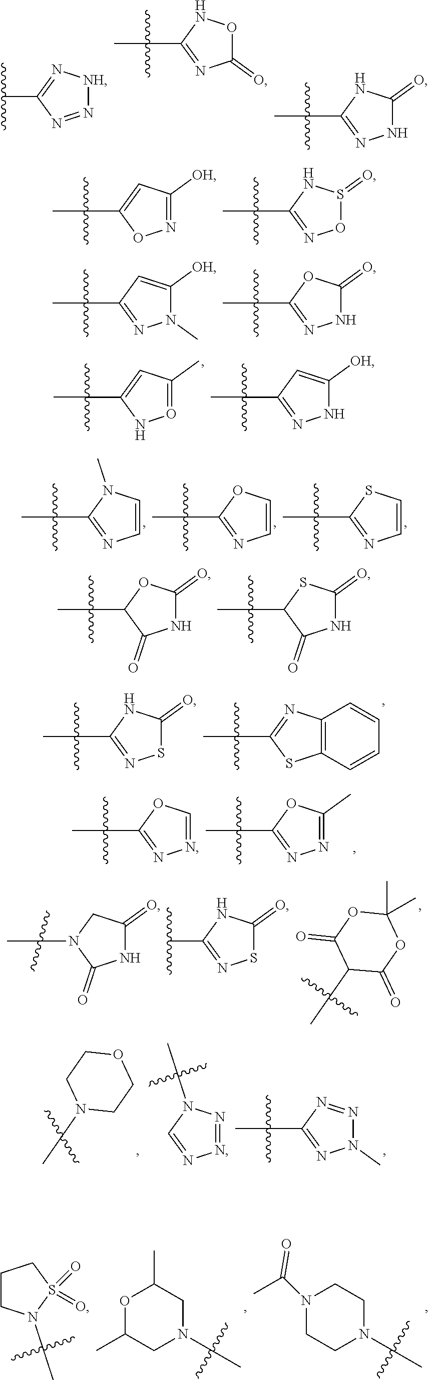

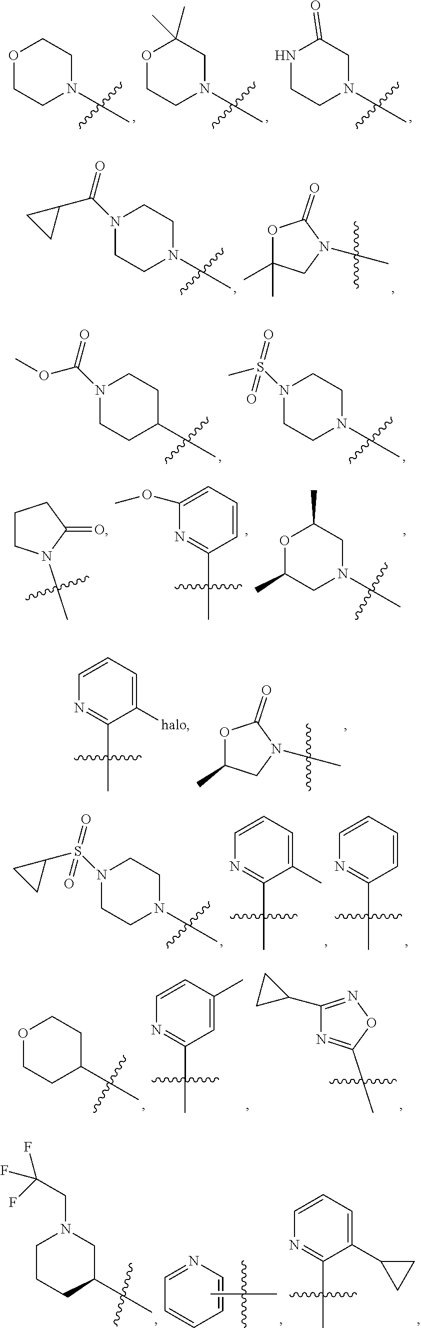

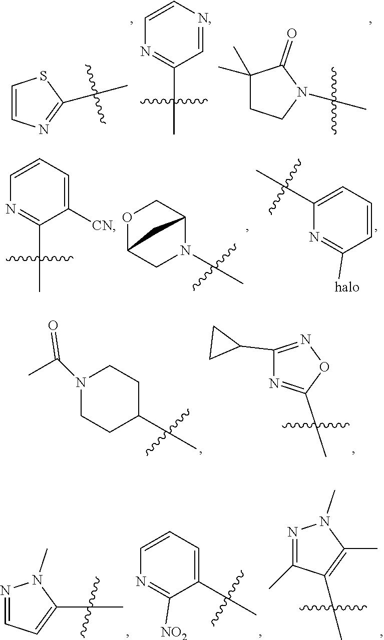

and the like.

The terms “carboxy” or “carboxyl,” whether used alone or with other terms, such as “carboxyalkyl,” denotes-CO₂H.

The term “carbonyl,” whether used alone or with other terms, such as “aminocarbonyl,” denotes —(C═O)—.

The term “cycloalkyl” includes saturated carbocyclic groups. Example of such radicals include, cyclopentyl, cyclopropyl, and cyclohexyl.

The term “cycloalkenyl” includes carbocyclic groups having one or more carbon-carbon double bonds including “cycloalkyldienyl” compounds.

The term “cholesteryl moiety” refers to the structure below:

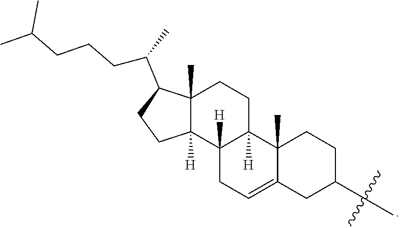

The wavy line: [symbol] indicates the connecting point.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

The symbol “-” represents a covalent bond and can also be used in a radical group to indicate the point of attachment to another group. In chemical structures, the symbol is commonly used to represent a methyl group in a molecule.

The term “excipient” means any pharmaceutically acceptable additive, carrier, diluent, adjuvant, or other ingredient, other than the active pharmaceutical ingredient (API), which is typically included for formulation and/or administration to a patient.

The term “therapeutically effective amount” means an amount of a compound that ameliorates, attenuates or eliminates one or more symptom of a particular disease or condition, or prevents or delays the onset of one of more symptom of a particular disease or condition.

The term “pharmaceutically acceptable” means that the referenced substance, such as a compound or composition described herein, or a salt thereof, or a formulation containing a compound described herein, or a particular excipient, are suitable for administration to a patient.

The terms “treating”, “treat” or “treatment” and the like include preventative (e.g., prophylactic) and palliative treatment.

As used herein, the term “about” means±10% of an associated numerical value.

All patents, published patent applications and other publications recited herein are hereby incorporated by reference.

Other aspects and embodiments of the invention will be apparent from the following Examples.

### EXAMPLES

The present teachings, having been generally described, will be more readily understood by reference to the following examples, which are included for the purposes of illustrating certain aspects and embodiments of the present disclosure.

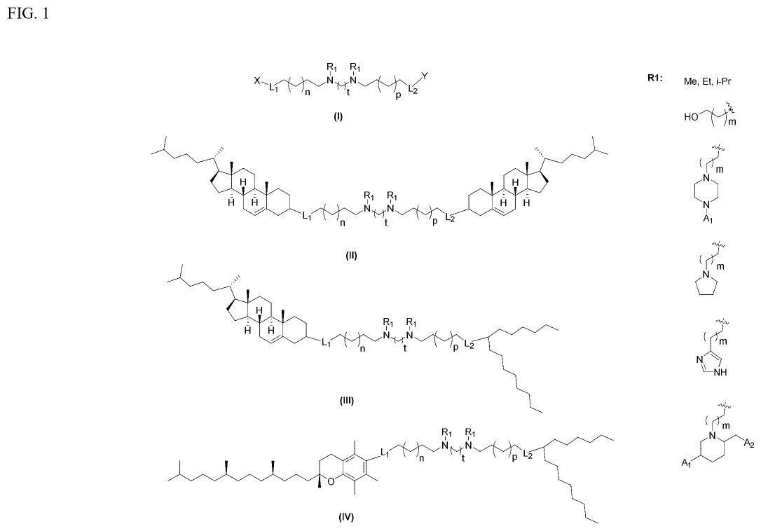

Messenger RNA (mRNA) has significant therapeutic potential, but continues to face limitation due to delivery vehicle efficiency. Effective formulations should safely shuttle mRNA, which is inherently unstable due to its poly-anionic nature, into the cytosol of target cells. In these examples, ionizable cationic lipids were designed and synthesized with two or more nitrogen atoms in the main chain. In accordance with embodiments of this disclosure, such lipids may allow for a reduction in the amount of lipids required for LNP formulation, compared to conventional lipid structures. These ionizable lipids were formulated via microfluidic mixing with three additional lipid components: 1,2-distearoyl-sn-glycero-3-phosphocholine (DSPC), cholesterol, and 1,2-dimyristoyl-rac-glycero-3-methoxypolyethylene glycol-2000 (DMG-PEG) at molar ratio of 50:10:38.5:1.5 with luciferase mRNA at N/P ratio 12. N/P is the ratio between amines in the ionizable lipid and the mRNA's anionic phosphates. The apparent pKa represents the lipid nanoparticle (LNP) surface ionization and is indirectly measured by the TNS assay. The LNP's apparent pKa correlates with mRNA delivery efficiency. Formulated LNPs were evaluated for particle size, size distribution range, and mRNA encapsulation efficiency. The following examples demonstrate a new class of ionizable lipids (embodiments of which are illustrated in FIG. 1) that exhibit efficient mRNA delivery to cells via LNPs.

### Example 1: General Synthetic Scheme

The following general synthetic scheme for embodiments of the invention is illustrated below for the synthesis of (Propane-1,3-diylbis(methylazanediyl))bis(heptane-7,1-diyl)bis(2-hexyldecanoate).

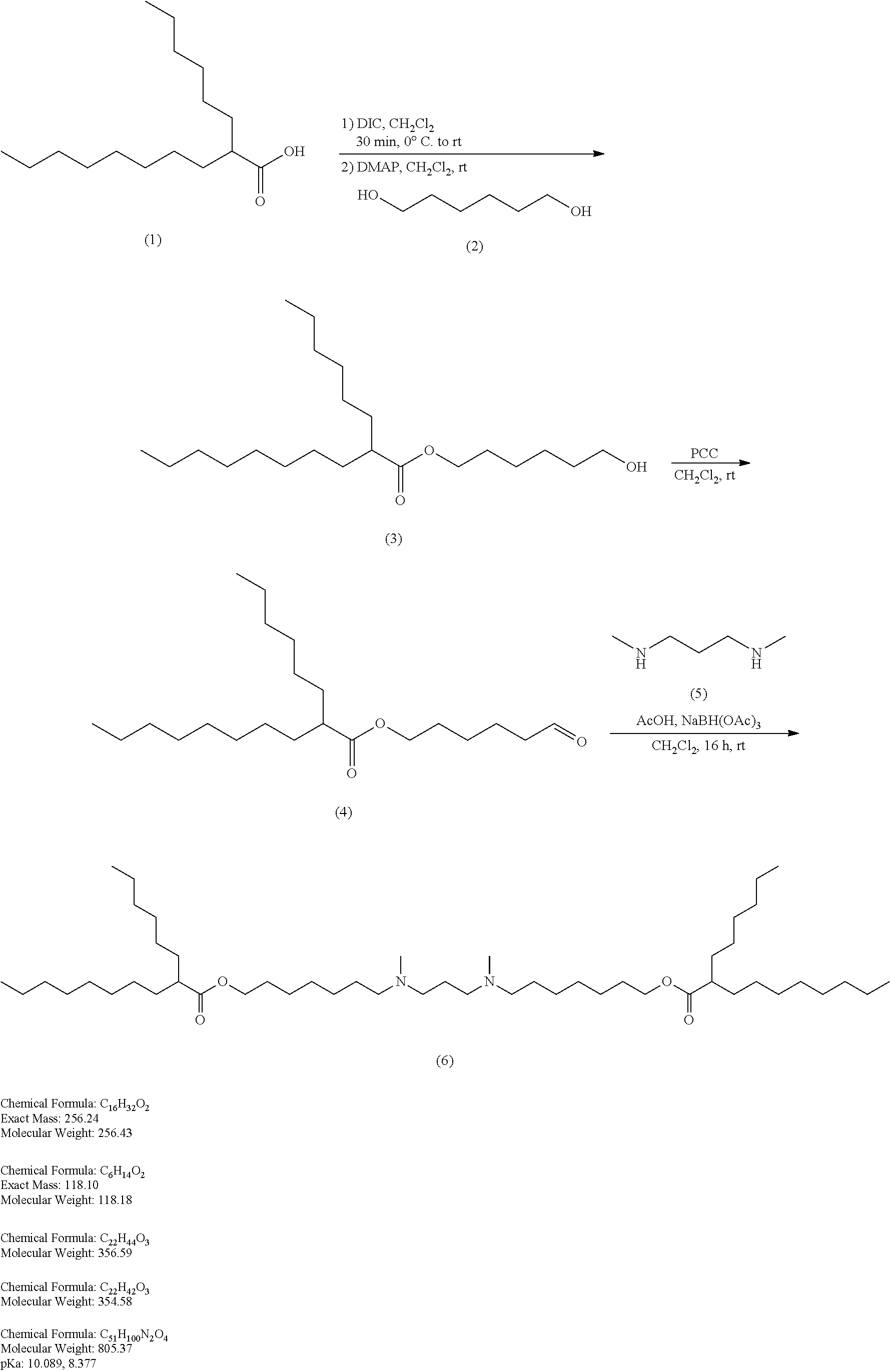

### 6-Hydroxyhexyl 2-Hexyldecanoate (3)

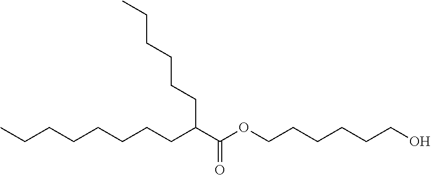 [SMILES: CCCCCCCCC(CCCCCC)C(=O)OCCCCCCO]

To a solution of 2-hexyldecanoic acid (1 eq) in anhydrous dichloromethane was added N,N′-diisopropylcarbodiimide (2 eq) under argon atmosphere on an ice bath (0° C.). The reaction mixture was stirred at 0° C. for 30 min and then 1,6-hexan-diol (1.05 eq) and DMAP (0.5 eq) were slowly added to the mixture. The reaction mixture was allowed to warm to room temperature and left for stirring overnight (16 h) at room temperature. The reaction mixture was washed with 30 mL saturated NaHCO₃ solution and extracted with 2×30 mL dichloromethane. The combined organic fractions were dried with anhydrous Na₂SO₄, filtered, and concentrated in vacuo. Purification by Teledyne ISCO NextGen300+silica flash chromatography (0-100% EtOAc in Hexane over 25 min) provided the desired product.

### 6-Oxohexyl 2-Hexyldecanoate (4)

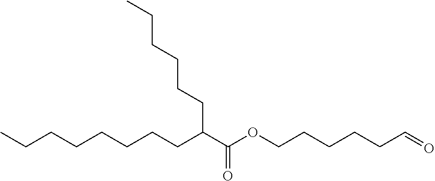 [SMILES: CCCCCCCCC(CCCCCC)C(=O)OCCCCCC=O]

To a solution of 6-hydroxyhexyl 2-hexyldecanoate (1 eq) in anhydrous dichloromethane was slowly added pyridinium chlorochromate (1.5 eq) over 10 min under argon atmosphere. The reaction mixture was stirred at room temperature for 2 h. The mixture was filtered through a celite pad and collected fractions were dried with anhydrous Na₂SO₄, filtered, and concentrated in vacuo. Purification by Teledyne ISCO NextGen300+silica flash chromatography (0-100% EtOAc in Hexane over 25 min) provided the desired product.

### (Propane-1,3-Diylbis(Methylazanediyl))Bis(Heptane-7,1-Diyl)Bis(2-Hexyldecanoate)(6)

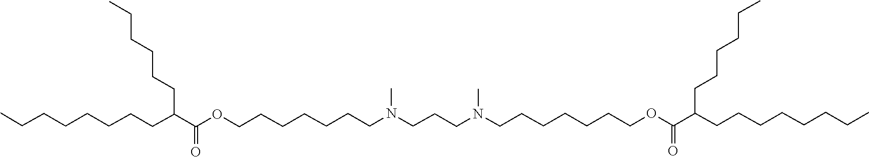 [SMILES: CCCCCCCCC(CCCCCC)C(=O)OCCCCCCCN(C)CCCN(C)CCCCCCCOC(=O)C(CCCCCC)CCCCCCCC]

To a solution of 6-oxohexyl 2-hexyldecanoate (3.0 eq) in anhydrous dichloromethane was added N,N-dimethyl-1,3 propanediamine (1.0 eq), sodium triacetoxy borohydride (3.0 eq) and few drops of acetic acid. The reaction mixture was stirred at room temperature under argon for 16 h. The reaction mixture was washed with 30 mL satd. NaHCO₃ solution and extracted with 2×30 mL dichloromethane. The combined organic fractions were dried with anhydrous Na₂SO₄, filtered, and concentrated in vacuo. Purification by Teledyne ISCO NextGen300+silica flash chromatography (0-20% MeOH in CH₂Cl₂ over 25 min) provided the desired product.

Lipids synthesized according to this general scheme are shown in Table 1.

### Example 2: LNP Formulation

Unless otherwise stated only RNAase and DNAase free materials were used. All the lipids stock solutions were warmed to 37° C. Stock solutions were visually inspected to make sure there were no crystals. Sonication (time varies) and/or heat gun (maximum 20 sec.) were used as necessary to dissolve any crystals. Luc-mRNA was thawed at 4° C. (no vortex or sonication). The lipid mix and ethanol were added to the vial (Organic phase). The mRNA and acetate buffer, pH 4, were combined to obtain the aqueous phase. After the addition of mRNA to the acidic buffer, the stock mRNA solution was returned to −80° C. freezer. The aqueous phase was loaded in a suitable syringe, avoiding any bubbles. Similarly, the organic phase was loaded into a separate syringe avoiding any bubbles.

The cartridge and 15 mL Falcon tube were inserted on NANOASSEMBLR Ignite (Precision NanoSystems). The formulation was made with the following settings: Flow rate ratio (FRR) 3:1, Total flow rate (TFR) 12 mL/min, Start waste 0.25 mL, and End waste 0.25 mL.

After pressing the start setting, the formulation was collected into the falcon tube, and it was diluted approximately 10X volume of PBS.

The formulation was transferred to the 10K Amicon filter (15 mL) and was centrifuged at 4° C. and 2000 rcf for 90 min or until the volume reduces to ˜400 μL. The formulation was collected, and it was characterized using DLS for size and PDI. The formulation was further analyzed for RNA encapsulation using Quant-iT™ RiboGreen® RNA assay kit.

The formulation was kept at 4° C. until used in the luciferase expression assay to evaluate LNP's potency, and for toxicity using LDH assay in the HeLa and HEK293 cells. LNPs were used in the TNS assay to determine the experimental (apparent) pKa of the ionizable lipid. Hemolysis assay was used to determine the indirect endosomal escape (EE) capability of the LNPs. LNPs were evaluated in vivo for luciferase expression 6 h and 24 h post-dose in mice through i.m. injection at 5 mg per mouse. Mice were sacrificed at the end point and then organs were harvested for ex-vivo imaging.

TABLE 2
Physiochemical properties of the LNPs.

| LNP | Size (nm) | PDI | EE |
|---|---|---|---|
| GILP-124 | 103 | 0.20 | 88% |
| GILP-126 | 78 | 0.12 | 93% |
| GILP-133 | 67 | 0.17 | 98% |

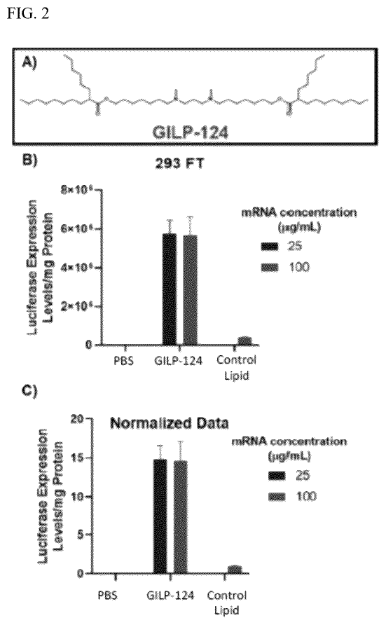

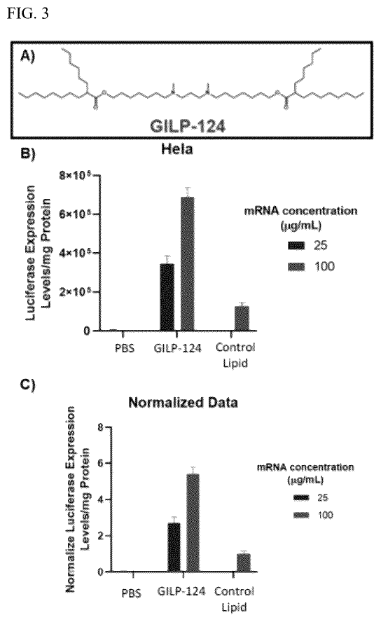

Lipid nanoparticles comprising GILP-124 (as described above and shown in FIGS. 2A and 3A) were tested for efficiency of mRNA delivery using HEK293 cells and HeLa cells. These studies employ encapsulation of luciferase mRNA. Results for HEK293 cells are shown in FIGS. 2B and 2C, against lipid nanoparticles containing a control ionizable lipid 47 eptadecane-9-yl 8-[2-hydroxyethyl-(6-oxo-6-undecoxyhexyl)amino]octanoate as shown in FIG. 7. FIG. 2C shows normalized values for the in vitro evaluation (i.e., setting the control value as 1 and calculating the relevant ratios for test samples on that basis). As shown, nanoparticles containing GILP-124 provided high efficiency of mRNA delivery to HEK293 cells, with even 25 μg/mL showing high levels of delivery. GLP-124 showed significant increase in efficiency vs. control. See FIGS. 2B and 2C. Similar results were obtained for HeLa cells, although the results were more dose-dependent for GILP-124 LNPs. See FIGS. 3B and 3C.

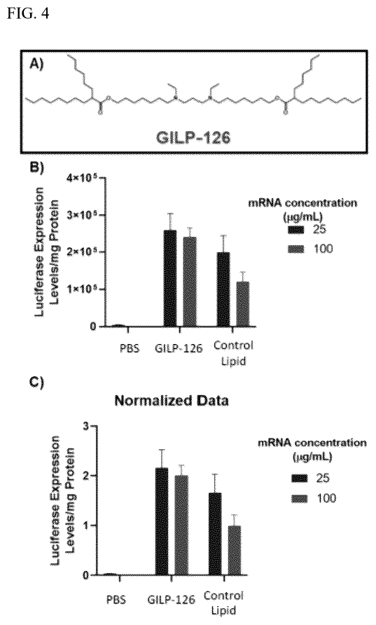

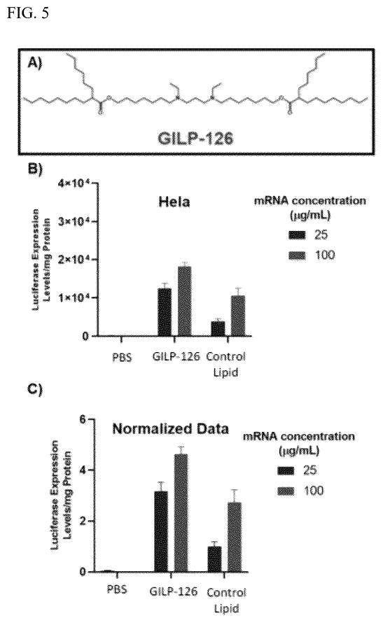

Lipid nanoparticles comprising GILP-126 (as described above and shown in FIGS. 4A and 5A) were similarly tested. Nanoparticles containing GILP-126 also performed substantially better than the positive control LNPs in both HEK293 and HeLa cells. See FIGS. 4B and 4C, and FIGS. 5B and 5C.

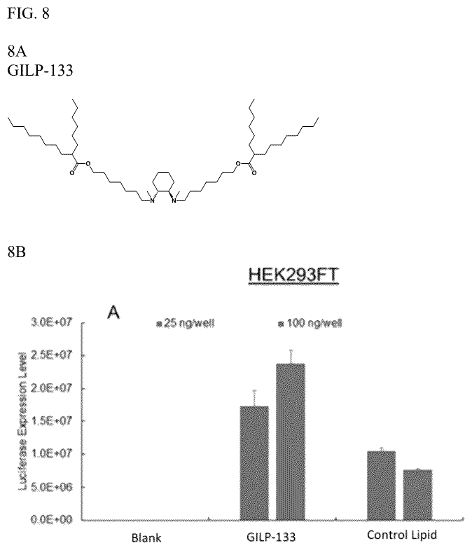

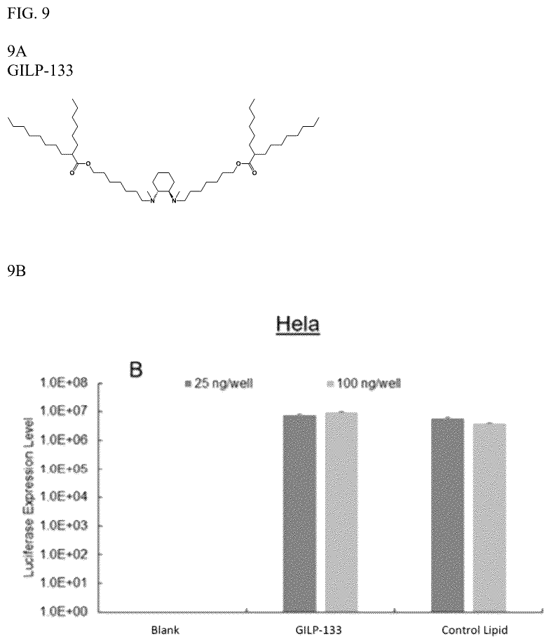

Lipid nanoparticles comprising GILP-133 (as described above and shown in FIGS. 8A and 9A) were similarly tested and found to perform substantially better than positive control LNPs in both HEK293 and HeLa cells. See FIGS. 8B and 9B.

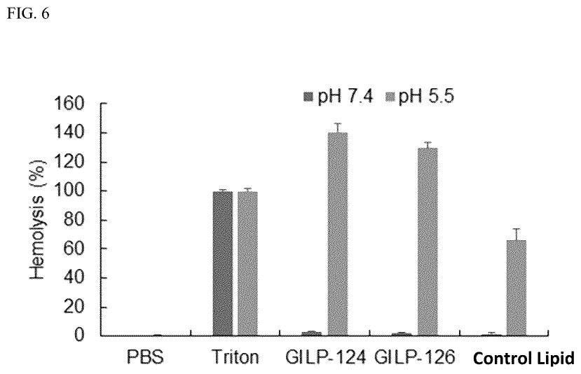

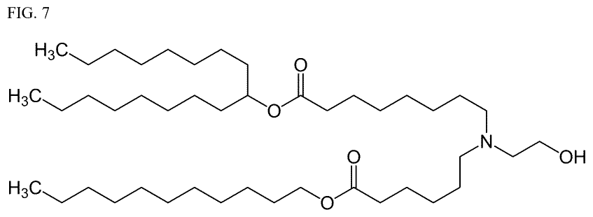

FIG. 6 illustrates endosomal escape ability of LNPs formulated using the ionizable lipids using a hemolysis assay. Remarkably, lipid nanoparticles formulated with either GILP-124 or GILP-126 exhibit negligible hemolysis at neutral pH (pH 7.4) (suggesting low toxicity), but exhibit strong hemolysis at acidic pH (pH 5.5) suggesting strong endocytic escape potential. LNPs comprising GILP-124 or GILP-126 appear to have substantially stronger endocytic escape potential as compared to control LNPs.

### Example 3. Animal Luciferase Expression Study

Two formulations containing GILP-133 (shown in Table 3) were selected to evaluate luciferase expression in vivo.

TABLE 3

| Formulation | N/P | Helper lipid | GILP:Helper:Cholesterol:DMG-PEG |
|---|---|---|---|
| 1 | 12 | DSPC | 50:10:38.5:1.5 |
| 2 | 12 | DSPC | 31.3:8.2:60:0.5 |
| 3 | 8 | DOPE | 23.4:16.1:60:0.5 |
| 4 | 12 | DSPC | 31.3:8.2:59:1.5 |

Each sample was prepared as described above in Example 1 and stored at −80° C. in the presence of 10% glycerol, 10% sucrose, 80% aqueous PBS. After freeze-thaw maintained acceptable particle size, PDI and EE, as shown in Table 4.

TABLE 4

| | Before F/T | | | After F/T | | |
|---|---|---|---|---|---|---|
| Formulation | Size (nm) | PDI | EE | Size (nm) | PDI | EE |
| 1 | 106 | 0.13 | 98% | 90 | 0.14 | 97% |
| 2 | 128 | 0.17 | 97% | 122 | 0.10 | 91% |
| 3 | 126 | 0.07 | 100% | 133 | 0.04 | 99% |
| 4 | 88 | 0.21 | 99% | 84 | 0.13 | 98% |

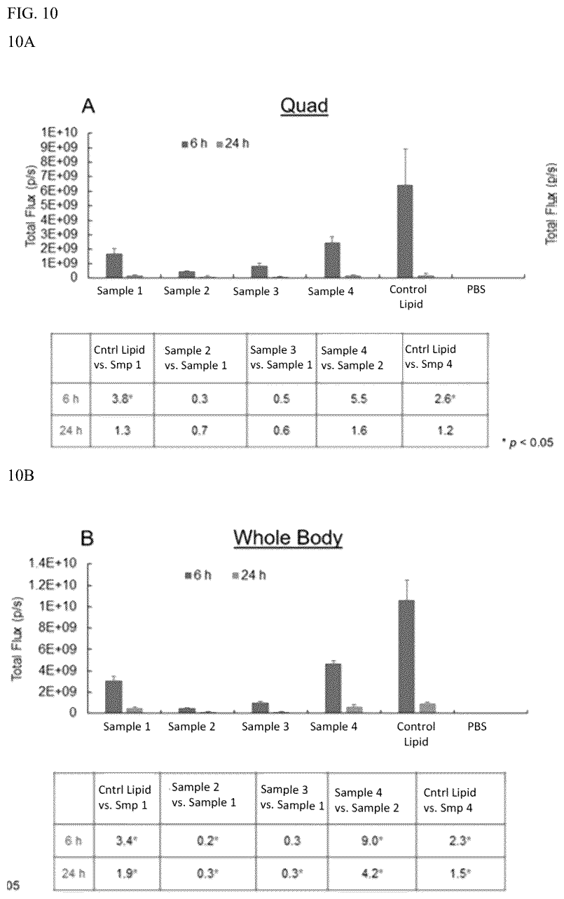

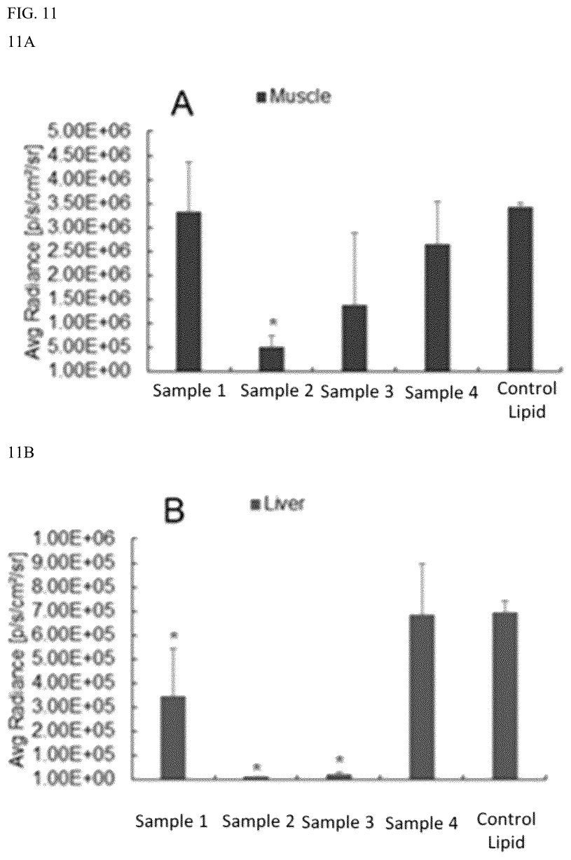

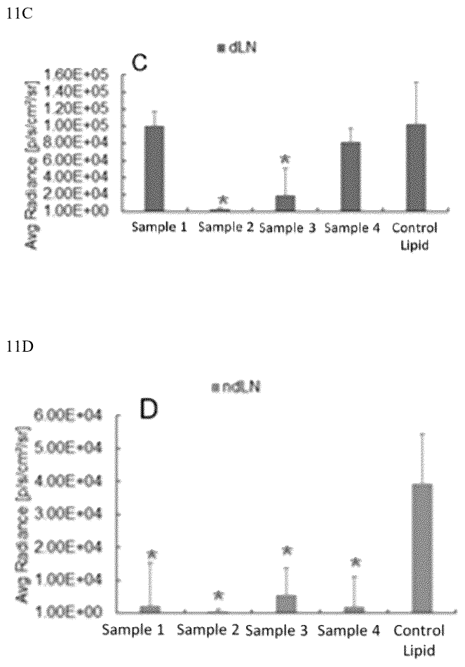

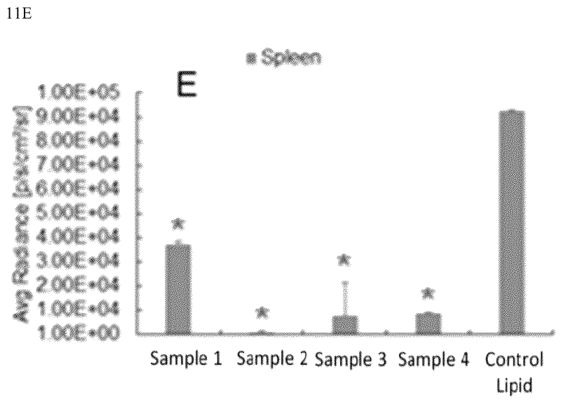

These samples were injected to mice through intramuscular injection at the leg site at a dose of 5 μg of mRNA per mouse. Compositions with control LNP of FIG. 7 were used as positive control. At 6 h and 24 h post injection, the mice were subjected to IVIS imaging system to take the whole-body image. At 6 h, Sample Nos. 2 and 3 showed lower luciferase expression to Sample No. 1 at the injection site, indicating poor in vitro and in vivo correlation. Sample 4 with higher ratio of DMG-PEG (1.5%) showed much higher luciferase expression compared to Sample 2 with lower ratio of DMG-PEG (0.5%). The same trend was found in the whole-body imaging results. Results are shown in FIGS. 10A and 10B.

At 24 h, the mice were sacrificed, and the main organs include heart, liver, spleen, lung, kidneys, muscle, dLNs and ndLNs were collected and imaged. Except for Sample No. 3, all the other GIL-133 LNP formulations showed comparable luciferase expression in the muscle. Sample Nos. 2 and 3 did not show any luciferase expressions in the liver, indicating that they may have the potential for beyond-liver delivery. In the dLNs, Sample Nos. 1 and 4 showed comparable luciferase expression level to positive control, indicating that they may have the potential to induce strong immune responses. In ndLNs and spleen, positive control showed higher level of luciferase expression to all GIL-133 formulations. All results are shown in 11A, 11B, 11C, 11D, and 11E.

### Example 4. Animal Immune Response Assay

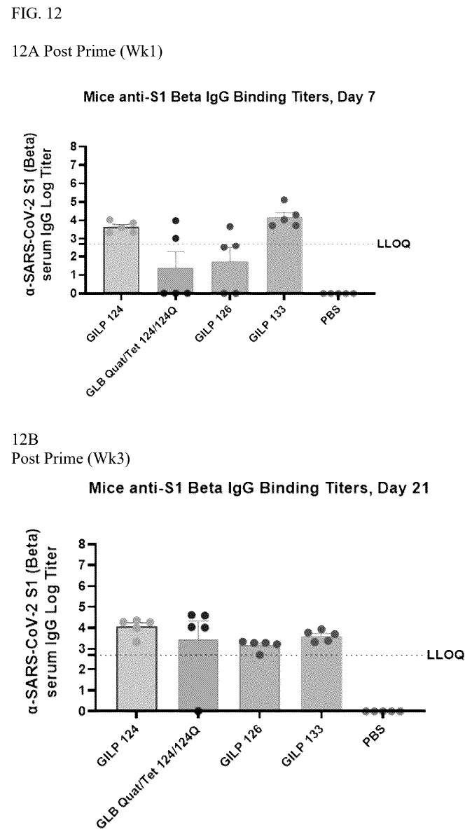

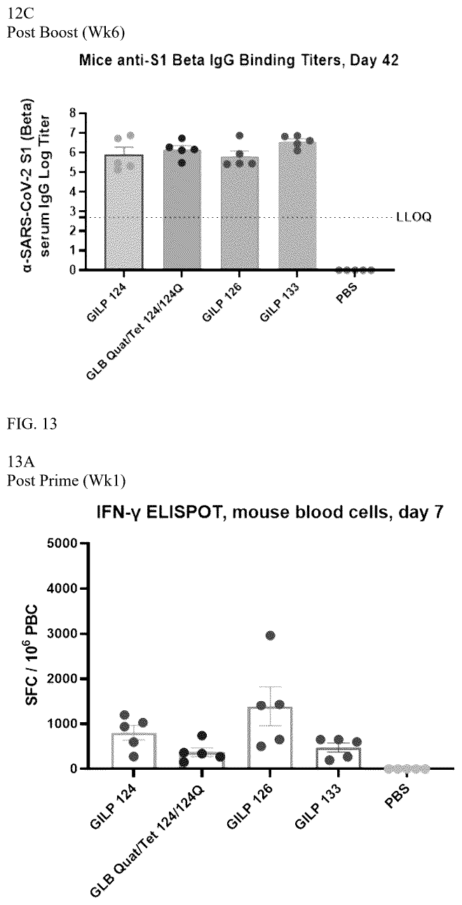

Vaccine formulations containing GILP 124, GILP 126, GILP 133, and GILP 124/124Q (a mixture of about 4:1 GILP124: GILP124Q: labeled as GLB Quat/Tet 124/124Q in FIG. 12 and FIG. 13) with the right nitrogen quaternary (positively charged)) were selected for study in a mouse immune response assay. mRNA encoding a SARS-COV-2 spike protein, beta variant were encapsulated in the LNP formulations. C₅₇BL/6 Mice were immunized with 5 μg of the vaccines on a regimen of prime and boost 21 days apart (prime day ( ) and boost day 21). The mice were euthanized on day 42. Blood was collected for the antibody and ELISpot analysis of antigen-specific IFN-γ producing T cells. Mice were separated into 5 groups, 5 mice per group (n=5).

### Anti-Beta SARS-COV-2 S1-Spike IgG Measurements by ELISA

From the blood collected on day 7, 21, and 42 ELISA were performed to evaluate seroconversion and IgG levels to the SARS-COV-2 Beta spike protein. On day 7, it was observed that not all animals seroconverted in the groups receiving the mRNA formulated into 124/124Q and GILP 126, but all animals seroconverted after a single dose of the mRNA when formulated into GILP 124 and GILP 133 as showed in FIG. 12A. On week 3, 21 days after the first dose all animals in all groups, except 1 mouse in the 124/124Q seroconverted with levels of anti-spike IgG ranging from 3.1 to 4.5 (in Log 10), circa 1.2-fold in Logio higher than the lower limit of quantification shown by the doted black line. (See FIG. 12B.) On day 42, 21 days after the boost dose of formulated mRNA the levels of anti-spike IgG, all animals in all groups seroconverted and showed high levels of anti-spike IgG. (See FIG. 12C) The results show that the tested formulations enabled the delivery of mRNA, which induced a strong antibody response against SARS-COV-2 Beta spike protein. End-point titers were calculated as the dilution that emitted an optical density exceeding 4x background (secondary antibody alone). All the measure values above the LLOQ were considered as anti-spike IgG positive in these assays.

Using a commercial ELISpot kit (BD™ ELISPOT Mouse IFN-γ ELISPOT Set) and following the vendors protocol, peripheral blood of mice was evaluated 7 days after the first dose of formulated 048 mRNA. Blood cells were lysed and plated overnight upon stimulation with a peptide pool of SARS-COV-2 Beta spike protein. An assay internal positive control was performed by using PMA/ION as stimuli. It was observed one dose of formulated mRNA was enough to prime antigen specific IFN-γ producing T cells as can be seen by the number of spots in FIG. 13A. The same assay was performed on day 42, 21 days after the boosting dose of mRNA in different formulations that the immune responses were several folds enhanced as showed by the number of spots in FIG. 13B. These data suggest that all such formulations successfully delivered the encapsulated antigen that induced a priming and boosting antigen specific T cells immune response measured by an IFN-γ ELISpot assay.

### Example 5—Alternative Structures to GILP-133

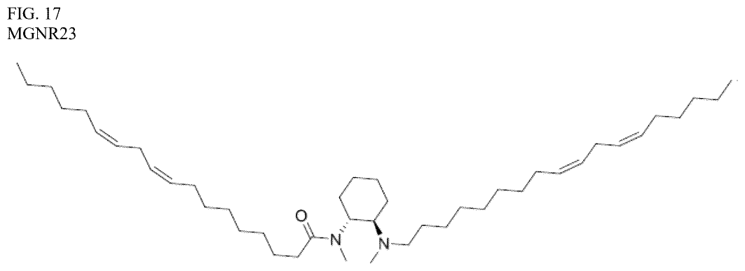

Alternative structures to GILP-133 were evaluated to consider whether a change to the number of linker carbons or a change to the ring structure at the center of the molecule would have any effect. MGNR 24 as shown in Table 1 differs from GILP-133 in incorporating 6-carbon linker regions rather than 7-carbon linkers region. MGNR23 as shown in FIG. 17 differed in that it incorporated lineolitic tails. And BCY-01 as shown in Table 1 differs in that it concorporated a bicyclic ring in L₃.

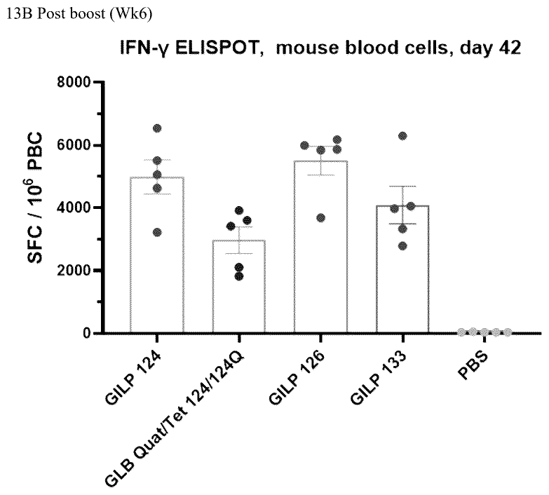

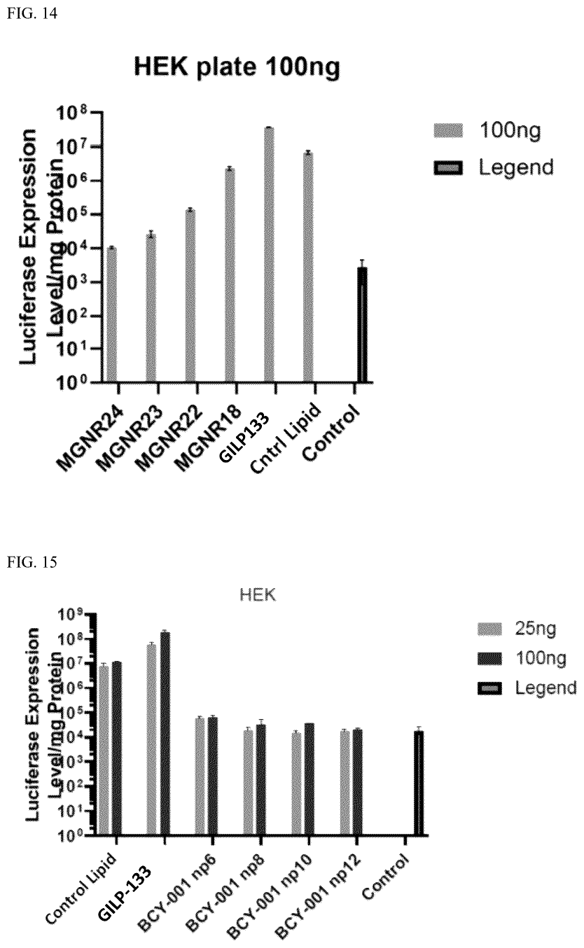

In HEK cell luciferase expression assays as described in Example 2, GILP-133 was superior to MGNR 24, MGNR 23, and various formulations comprising BCY-01. See FIGS. 14 and 15.

### Example 6—LDH Toxicity Assay

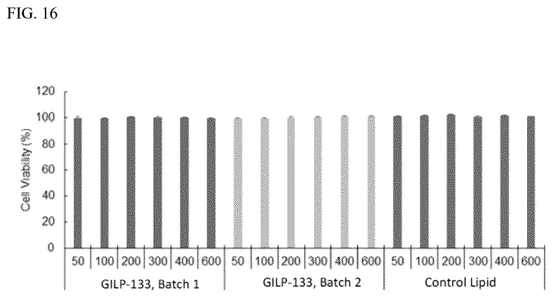

GILP-133 was tested in an assay to measure the level of lactate dehydrogenase (LDH), also known as lactic acid dehydrogenase. The LDH assay protocol is based on an enzymatic coupling reaction: LDH released from the cell oxidizes lactate to generate NADH, which then reacts with WST to generate a yellow color. The intensity of the generated color correlates directly with the number of lysed cells. This gives an indication of cytotoxicity, looking at the % of viable cells remaining. In this testing, Hek 293 FT cells were used 10 uL 1% Triton was added to positive control wells and incubated for 5 min. 50 uL cell culture medium was transferred to black 96-well plate. 50 uL LDH assay solution is added and incubated for 10 min at room temperature. Subsequently 50 ul stop solution was added and fluorescence intensity was read using a microplate reader. As shown in FIG. 16, GILP-133 from two separate batches showed equivalent % cell viability compared to control cationic lipid shown in FIG. 7. This suggests that GILP-133 is not more toxic at the tested concentrations than a control lipid already approved for use in humans.

TABLE 1
Examples of Ionizable Lipids

| | | | pKa | |
|---|---|---|---|---|
| | | | (Chem- | |
| Com- | | | Draw | |
| pound | | IUPAC | Calcu- | |
| No. | Structure | Name | lated) | Characterization |
| GILP- 124 | 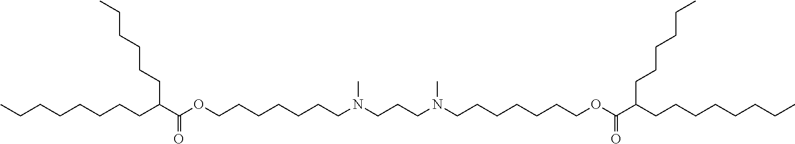 [SMILES: CCCCCCCCC(CCCCCC)C(=O)OCCCCCCCN(C)CCCN(C)CCCCCCCOC(=O)C(CCCCCC)CCCCCCCC] | (propane- 1,3-diylbis (methyl- azanediyl)) bis(heptane- 7,1-diyl) bis(2-hexyl- decanoate) | 8.615 | ¹H NMR (400 MHz, CDCl₃) δ(ppm): 4.05 (t, J = 6.7 Hz, 4H), 2.55-2.40 (m, 7H), 2.35- 2.24 (m, 8H), 1.65-1.49 (m, 12H), 1.48-1.38 (m, 5H), 1.33 (dt, J = 8.6, 5.4 Hz, 16H), 1.25 (s, 42H), 0.87 (t, J = 6.7 Hz, 12H). ¹³C NMR (101 MHz, CDCl₃) δ(ppm): 176.9, 64.2, 57.6, 55.4, 46.0, 32.7, 32.0, 31.9, 29.7, 29.6, 29.4, 29.3, 29.2, 28.8, 27.6, 27.5, 27.4, 26.1, 22.8, 22.7, 14.3, 14.2 |
| GILP- 126 | 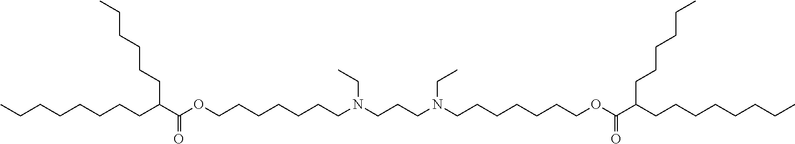 [SMILES: CCCCCCCCC(CCCCCC)C(=O)OCCCCCCCN(CC)CCCN(CC)CCCCCCCOC(=O)C(CCCCCC)CCCCCCCC] | (propane- 1,3-diylbis (ethyl- azanediyl)) bis(heptane- 7,1-diyl) bis(2-hexyl- decanoate) | 8.656 | ¹H NMR (400 MHz, CDCl₃) δ(ppm): 4.06 (t, J = 6.7 Hz, 4H), 2.58 (d, J = 43.8 Hz, 11H), 2.30 (tt, J = 8.9, 5.3 Hz, 2H), 1.67-1.47 (m, 13H), 1.46-1.36 (m, 6H), 1.33 (dt, J = 9.5, 5.8 Hz, 11H), 1.26 (s, 42H), 1.09 (t, J = 7.2 Hz, 6H), 0.87 (t, J = 6.7 Hz, 12H). ¹³C NMR (101 MHz, CDCl₃) δ(ppm): 176.9, 64.2, 47.4, 46.0, 32.7, 32.0, 31.9, 29.7, 29.6, 29.4, 29.3, 29.3, 28.8, 27.6, 27.5, 26.1, 22.8, 22.7, 14.3, 14.2 |
| GILP- 130 | 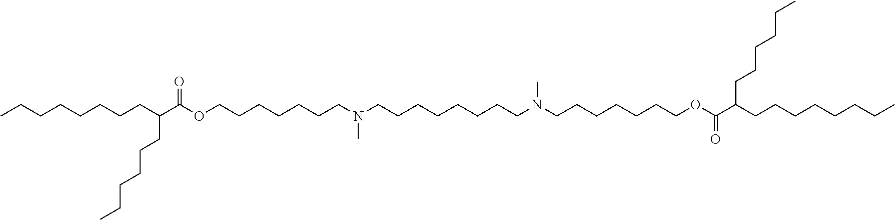 [SMILES: CCCCCCCCC(CCCCCC)C(=O)OCCCCCCCN(C)CCCCCCCCN(C)CCCCCCCOC(=O)C(CCCCCC)CCCCCCCC] | (octane-1,8- diylbis (methyl- azanediyl)) bis(heptane- 7,1-diyl) bis(2-hexyl- decanoate) | 10.192 | ¹H NMR (400 MHz, CDCl₃) δ(ppm): 4.06 (t, J = 6.6 Hz, 4H), 2.34-2.25 (m, 10H), 2.19 (s, 6H), 1.67 (s, 3H), 1.64-1.52 (m, 8H), 1.50-1.37 (m, 14H), 1.36-1.28 (m, 22H), 1.27-1.18 (m, 38H), 0.91-0.82 (m, 12H). ¹³C NMR (101 MHz, CDCl₃) δ(ppm): 176.9, 77.5, 77.2, 76.8, 64.3, 58.2, 58.1, 46.0, 42.5, 32.7, 32.0, 31.9, 29.8, 29.7, 29.6, 29.4, 29.3, 28.8, 27.8, 27.7, 27.6, 27.5, 27.5, 27.4, 26.1, 22.8, 22.7, 14.3, 14.2 |
| GILP- 132 | 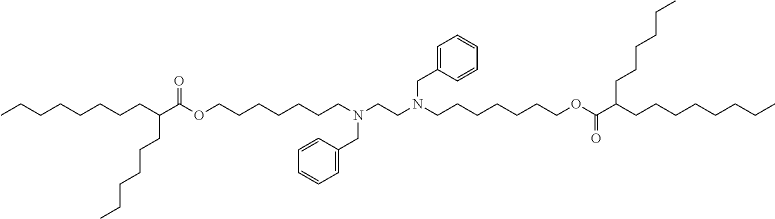 [SMILES: CCCCCCCCC(CCCCCC)C(=O)OCCCCCCCN(CCN(CCCCCCCOC(=O)C(CCCCCC)CCCCCCCC)Cc1ccccc1)Cc1ccccc1] | (ethane-1,2- diylbis (benzyl- azanediyl)) bis(heptane- 7,1-diyl) bis(2-hexyl- decanoate) | 6.326 | ¹H NMR (400 MHz, CDCl₃) δ(ppm): 7.27 (s, 2H), 7.25 (d, J = 2.9 Hz, 2H), 7.21 (td, J = 4.8, 2.8 Hz, 2H), 4.04 (t, J = 6.7 Hz, 4H), 3.54 (s, 3H), 2.54 (s, 4H), 2.41-2.33 (m, 4H), 2.30 (dq, J = 8.9, 4.5 Hz, 2H), 1.64-1.52 (m, 9H), 1.47- 1.35 (m, 9H), 1.36-1.14 (s, 55H), 0.87 (td, J = 6.9, 1.7 Hz, 12H). ¹³C NMR (101 MHz, CDCl₃) δ(ppm): 176.9, 139.9, 129.0, 128.2, 126.9, 64.3, 59.1, 54.4, 51.8, 46.0, 32.7, 32.0, 31.9, 29.7, 29.6, 29.4, 29.4, 29.3, 28.8, 28.7, 27.6, 27.5, 27.4, 27.1, 26.1, 22.8, 22.7, 14.3, 14.2 |
| GILP- 136 | 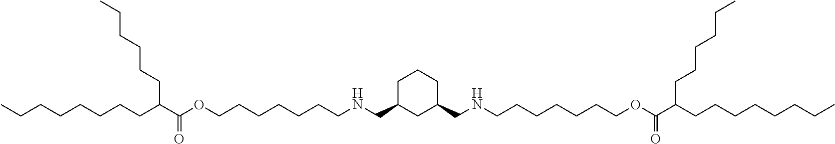 [SMILES: CCCCCCCCC(CCCCCC)C(=O)OCCCCCCCNC[C@H]1CCC[C@@H](CNCCCCCCCOC(=O)C(CCCCCC)CCCCCCCC)C1] | ((((1R,3S)- cyclohexane- 1,3-diyl)bis (methylene)) bis (azanediyl)) bis (heptane- 7,1-diyl) bis(2-hexyl- decanoate) | | |
| GILP- 138 | 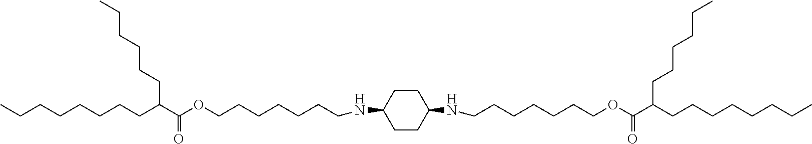 [SMILES: CCCCCCCCC(CCCCCC)C(=O)OCCCCCCCN[C@H]1CC[C@@H](NCCCCCCCOC(=O)C(CCCCCC)CCCCCCCC)CC1] | (((1s,4s)- cyclohexane- 1,4-diyl)bis (azanediyl)) bis(heptane- 7,1-diyl) bis(2-hexyl- decanoate) | | |
| GILP- 140 | 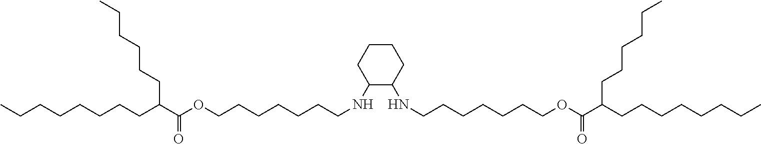 [SMILES: CCCCCCCCC(CCCCCC)C(=O)OCCCCCCCNC1CCCCC1NCCCCCCCOC(=O)C(CCCCCC)CCCCCCCC] | (cyclohexane- 1,2-diylbis (azanediyl)) bis(heptane- 7,1-diyl) bis(2-hexyl- decanoate) | | |
| GILP- 133 | 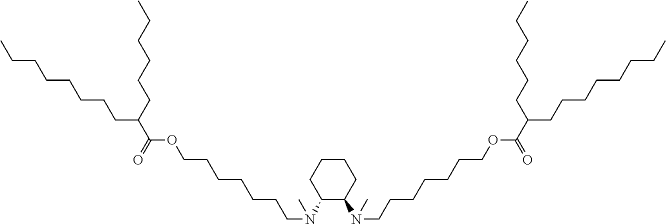 [SMILES: CCCCCCCCC(CCCCCC)C(=O)OCCCCCCCN(C)[C@@H]1CCCC[C@H]1N(C)CCCCCCCOC(=O)C(CCCCCC)CCCCCCCC] | (((1R,2S)- cyclohexane- 1,2-diyl)bis (methyl- azanediyl)) bis(heptane- 7,1-diyl) bis(2-hexyl- decanoate) | 6.957 | ¹H NMR (400 MHz, CDCl₃) δ(ppm): 4.06 (t, J = 6.7 Hz, 4H), 2.46 (d, J = 17.0 Hz, 6H), 2.30 (tt, J = 8.9, 5.4 Hz, 3H), 2.23 (s, 5H), 2.04 (d, J = 5.4 Hz, 1H), 1.65-1.52 (m, 9H), 1.50-1.39 (m, 8H), 1.37- 1.28 (m, 15H), 1.25 (s, 38H), 1.14-1.04 (m, 3H), 0.87 (t, J = 6.6 Hz, 12H). ¹³C NMR (101 MHz, CDCl₃) δ(ppm): 176.9, 64.3, 63.1, 58.6, 46.0, 36.7, 32.7, 32.0, 31.9, 29.7, 29.6, 29.5, 29.4, 29.3, 28.9, 27.8, 27.6, 27.5, 26.2, 22.8, 22.8, 14.3, 14.2. |
| MGNR- 24 | 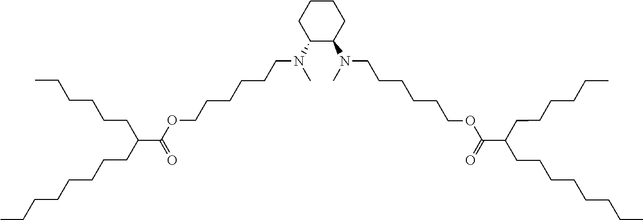 [SMILES: CCCCCCCCC(CCCCCC)C(=O)OCCCCCCN(C)[C@@H]1CCCC[C@H]1N(C)CCCCCCOC(=O)C(CCCCCC)CCCCCCCC] | (((1R,2R)- cyclohexane- 1,2-diyl)bis (methyl- azanediyl)) bis(pentane- 5,1-diyl) bis(2-hexyl- decanoate) | | |
| GILP- 134 | 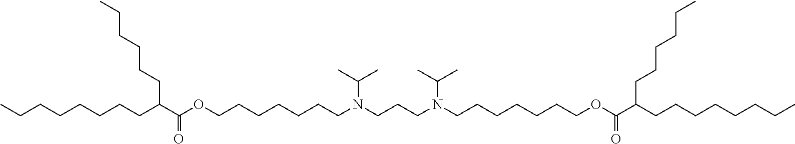 [SMILES: CCCCCCCCC(CCCCCC)C(=O)OCCCCCCCN(CCCN(CCCCCCCOC(=O)C(CCCCCC)CCCCCCCC)C(C)C)C(C)C] | (propane- 1,3-diylbis (isopropyl- azanediyl)) bis(heptane- 7,1-diyl) bis(2-hexyl- decanoate) | | |
| GILP- 135 | 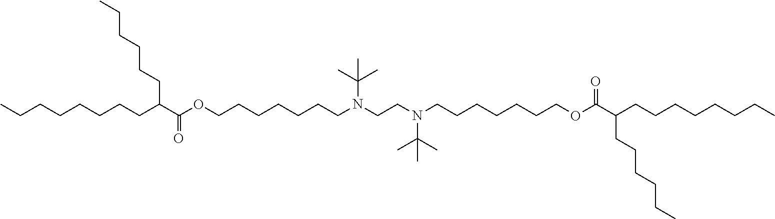 [SMILES: CCCCCCCCC(CCCCCC)C(=O)OCCCCCCCN(CCN(CCCCCCCOC(=O)C(CCCCCC)CCCCCCCC)C(C)(C)C)C(C)(C)C] | (ethane-1,2- diylbis(tert- butyl- azanediyl)) bis(heptane- 7,1-diyl) bis(2-hexyl- decanoate) | | |
| BCY- 01 | 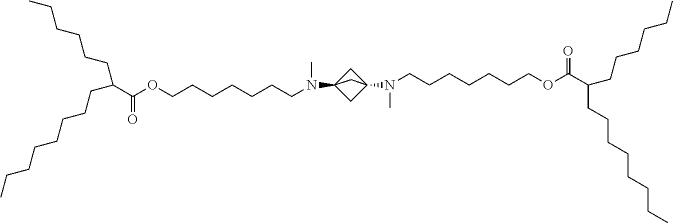 [SMILES: CCCCCCCCC(CCCCCC)C(=O)OCCCCCCCN(C)[C@]12C[C@@](N(C)CCCCCCCOC(=O)C(CCCCCC)CCCCCCCC)(C1)C2] | (bicyclo [1.1.1] pentane- 1,3-diylbis (methyl- azanediyl)) bis(heptane- 7,1-diyl) bis(2-hexyl- decanoate) | | |
| GIL- 137 | 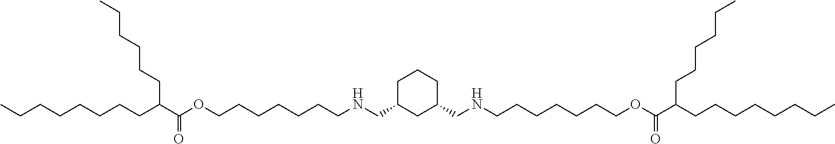 [SMILES: CCCCCCCCC(CCCCCC)C(=O)OCCCCCCCNC[C@H]1CCC[C@@H](CNCCCCCCCOC(=O)C(CCCCCC)CCCCCCCC)C1] | ((((1R,3S)- cyclohexane- 1,3-diyl)bis (methylene)) bis (azanediyl)) bis(heptane- 7,1-diyl) bis(2-hexyl- decanoate) | | |

1. A lipid nanoparticle composition comprising an ionizable lipid of Formula (I): 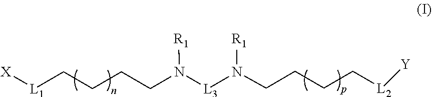
wherein:
each R₁ independently is H or a substituent;
L₁ is —OC(═O)— or —C(═O)O—;
L₂ is —OC(═O)— or —C(═O)O—;
L₃ is selected from the group consisting of: 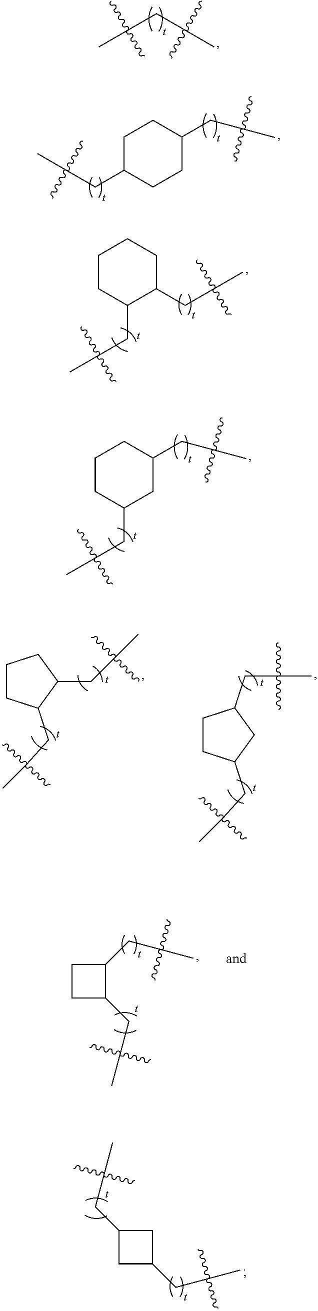
each of n, t, and p is independently selected from 0, 1, 2, 3, 4, 5, 6, 7, 8, 9, and 10;
and X and Y are each independently selected lipophilic moieties having at least six carbon atoms.

2. The lipid nanoparticle composition of claim 1, wherein each R₁ is independently selected from: H, (C₁-C₆)alkyl, substituted (C₁-C₆)alkyl, (C₁-C₆)alkenyl, substituted (C₁-C₆)alkenyl, cycloalkyl, cycloalkenyl, aryl, heteroaryl, and heterocyclo.

3. The lipid nanoparticle composition of claim 1 or claim 2, wherein each R₁ is independently selected from (halo)(C₁-C₆)alkyl, (alkoxy)(C₁-C₆)alkyl, (hydroxy)(C₁-C₆)alkyl, —(CH₂)ₙ—S—(C₁-C₆)alkyl, —(CH₂)ₙ—O—(C₁-C₆)alkyl, —C(O)(C₁-C₆)alkyl, (C₃-C₁₂)cycloalkyl, and (C₃-C₁₂)cycloalkenyl, any of which is optionally independently substituted as allowed by valence.

4. The lipid nanoparticle composition of claim 1 or 2, wherein each R₁ is independently selected from —(CH₂)ₙCHZR′, —CHZR′, —CZ(R′)₂, and —(CH₂)ₙZ, wherein Z is selected from —OC(O)(C₁-C₆)alkyl, —C(O)O(C₁-C₆)alkyl, —OC(O)(C₁-C₆)alkenyl, —C(O)O(C₁-C₆)alkenyl, (C₃-C₁₂)cycloalkyl, and (C₃-C₁₂)cycloalkenyl, any of which is optionally independently substituted as allowed by valence: wherein R′ is a substituent.

5. The lipid nanoparticle composition of claim 4, wherein R′ at each occurrence is independently halo, hydroxyl, cyano, nitro, oxo, (C₁-C₆)alkyl, (C₁-C₆)alkoxy, (C₁-C₆) haloalkyl, (C₂-C₆)alkenyl, (C₁-C₆)alkynyl, (C₃-C₁₂)cycloalkyl, (C₃-C₁₂)cycloalkenyl, heterocyclo, aryl, or heteroaryl.

6. The lipid nanoparticle composition of claim 1, wherein each R₁ independently is a C₁ to C₃ alkyl, and which is optionally methyl, ethyl or isopropyl.

7. The lipid nanoparticle composition of claim 4, wherein each R₁ independently is —(CH₂)ₙZ, and Z is aryl or heterocyclo.

8. The lipid nanoparticle composition of claim 7, wherein each R₁ independently is —(CH₂)ₙZ, and each Z is selected from phenyl, morpholinyl, pyrrolidinyl, imidazolidinyl, imidazolyl, pyrazolidinyl, pyrazolyl, oxazolidinyl, oxazolyl, pyridinyl, piperidinyl, diazinanyl, and diazinyl.

9. The lipid nanoparticle composition of claim 1, wherein each R₁ is independently selected from the group consisting of: 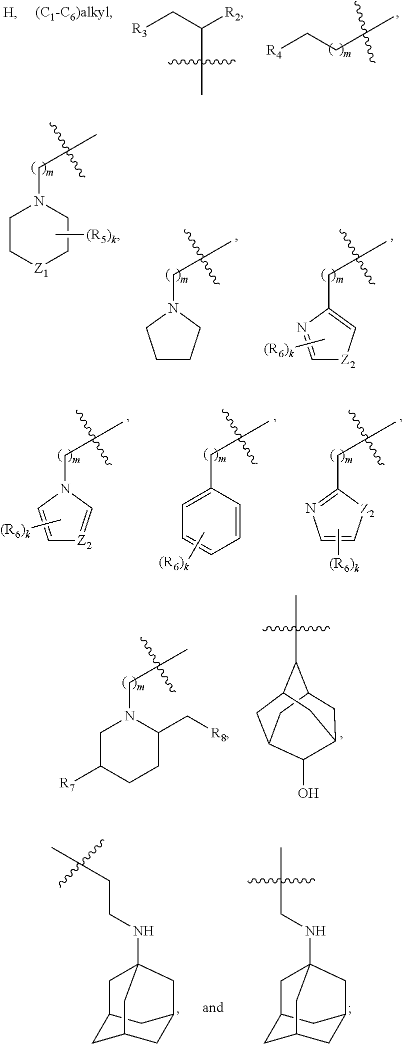
wherein: each m is independently 0, 1, 2, 3, 4, 5, or 6;
each k is independently 0, 1, 2, 3, or 4;
R₂ is CH₂OH or CO₂ (C₁-C₆)alkyl;
R₃ is OH, (C₁-C₆)alkyl, or aryl;
R₄ is OH, O(C₁-C₆)alkyl, SH, or S(C₁-C₆)alkyl;
each R₅ independently is H, (C₁-C₆)alkyl, —(CH₂) OH, or O(C₁-C₆)alkyl;
each R₆ independently is H, (C₁-C₆)alkyl, —(CH₂) OH, or O(C₁-C₆)alkyl;
R₇ is H or (C₁-C₆)alkyl;
R& is H or (C₁-C₆)alkyl;
Z₁ is NH, O, CH₂, or NRs;
Z₂ is O, S, NR₆, N, or NH.

10. The lipid nanoparticle composition of any one of claims 1 to 9, wherein X and Y are independently selected from linear or branched alkyl, linear or branched alkenyl, sterol, polyphenol, flavonoid, and tocopherol.

11. The lipid nanoparticle composition of claim 10, wherein one or both of X and Y are cholesteryl ester.

12. The lipid nanoparticle composition of claim 10, wherein one or both of X and Y are ester of tocopherol, which is optionally alpha, beta, gamma, or delta.

13. The lipid nanoparticle composition of claim 10, wherein one or both of X and Y are a branched alkyl or alkenyl having from 8 to 30 carbon atoms, and optionally from 10 to 20 carbon atoms.

14. The lipid nanoparticle composition of claim 13, wherein one or both of X and Y is:

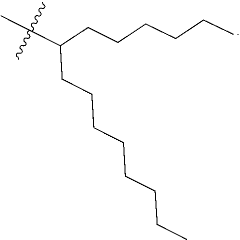 [SMILES: *C(CCCCCC)CCCCCCCC]

15. The lipid nanoparticle composition of claim 10, wherein X and/or Y is a flavonoid, and which is optionally selected from quercetin, rutin, macluraxanthone, genistein, scopoletin, daidzein, taxifolin, naringenin, abyssinones, eriodictyol, fisetin, theaflavin, peonidin, diosmetin, tricin, biochanin, hesperidin, epicatechin, myricetin, kaempferol, luteolin, and apigenin.

16. The lipid nanoparticle composition of claim 10, wherein X is cholesteryl ester and Y is

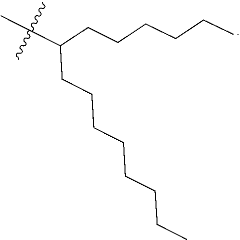 [SMILES: *C(CCCCCC)CCCCCCCC]

17. The lipid nanoparticle composition of claim 10, wherein X is tocopherol, and which is optionally alpha-tocopherol, and Y is:

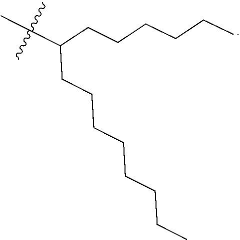 [SMILES: *C(CCCCCC)CCCCCCCC]

18. The lipid nanoparticle composition of any one of claims 1 to 17, wherein t is from 2 to 5, and optionally 3.

19. The lipid nanoparticle composition of any one of claims 1 to 18, wherein n and p are each an integer in the range of 3 to 10, and optionally 6, 7, or 8.

20. The lipid nanoparticle composition of claim 1 selected from the group consisting of:
(propane-1,3-diylbis(methylazanediyl))bis(heptane-7,1-diyl)bis(2-hexyldecanoate),
(propane-1,3-diylbis(ethylazanediyl))bis(heptane-7,1-diyl)bis(2-hexyldecanoate),
(octane-1,8-diylbis(methylazanediyl))bis(heptane-7,1-diyl)bis(2-hexyldecanoate),
(ethane-1,2-diylbis(benzylazanediyl))bis(heptane-7,1-diyl)bis(2-hexyldecanoate),
((((1R,3S)-cyclohexane-1,3-diyl)bis(methylene))bis(azanediyl))bis(heptane-7,1-diyl)bis(2-hexyldecanoate),
(((1s,4s)-cyclohexane-1,4-diyl)bis(azanediyl)) bis(heptane-7,1-diyl)bis(2-hexyldecanoate),
(cyclohexane-1,2-diylbis(azanediyl))bis(heptane-7,1-diyl)bis(2-hexyldecanoate),
(((1R,2S)-cyclohexane-1,2-diyl)bis(methylazanediyl))bis(heptane-7,1-diyl)bis(2-hexyldecanoate),
(((1R,2R)-cyclohexane-1,2-diyl)bis(methylazanediyl))bis(pentane-5,1-diyl)bis(2-hexyldecanoate),
(propane-1,3-diylbis(isopropylazanediyl))bis(heptane-7,1-diyl)bis(2-hexyldecanoate),
(ethane-1,2-diylbis(tert-butylazanediyl))bis(heptane-7,1-diyl)bis(2-hexyldecanoate),
(bicyclo[1.1.1]pentane-1,3-diylbis(methylazanediyl))bis(heptane-7,1-diyl)bis(2-hexyldecanoate), and
((((1R,3S)-cyclohexane-1,3-diyl)bis(methylene))bis(azanediyl)) bis(heptane-7,1-diyl)bis(2-hexyldecanoate).

21. The lipid nanoparticle composition of claim 1, wherein the ionizable lipid is (((1R,2S)-cyclohexane-1,2-diyl)bis(methylazanediyl))bis(heptane-7,1-diyl)bis(2-hexyldecanoate).

22. The lipid nanoparticle composition of any one of claims 1 to 21, further comprising one or more structural lipids.

23. The lipid nanoparticle composition of claim 22, wherein the structural lipid is selected from one or more of cholesterol, fecosterol, sitosterol, ergosterol, campesterol, stigmasterol, brassicasterol, tomatidine, ursolic acid, alpha-tocopherol, and mixtures thereof.

24. The lipid nanoparticle composition of claim 23, wherein the structural lipid is cholesterol.

25. The lipid nanoparticle composition of any one of claims 1 to 24, further comprising one or more phospholipids.

26. The lipid nanoparticle composition of claim 25, wherein the phospholipid is one or more selected from 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OchemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

27. The lipid nanoparticle composition of any one of claims 1 to 26, further comprising one or more conjugated lipids that inhibit aggregation of particles, optionally wherein the one or more conjugated lipids that inhibit aggregation of particles comprises one or more PEG lipids.

28. The lipid nanoparticle composition of claim 27, wherein the one or more PEG lipids is selected from one or more of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

29. The lipid nanoparticle composition of claim 28, wherein the one or more PEG lipids is 1,2-dimyristoyl-rac-glycero-3-methoxypolyethylene glycol-2000 (DMG-PEG).

30. The lipid nanoparticle composition of any one of claims 1 to 29, comprising a structural lipid, a PEG lipid, and a phospholipid.

31. The lipid nanoparticle composition of claim 30, wherein the molar ratio of the compound of Formula 1 over the phospholipid is in the range of about 1:1 to about 10:1: or the range of about 2:1 to about 9:1: or the range of about 3:1 to about 8:1: or the range of about 4:1 to about 7:1: or the range of about 4:1 to about 6:1, and is optionally about 5:1.

32. The lipid nanoparticle composition of claim 30 or 31, wherein the molar ratio of the phospholipid over the structural lipid is in the range of about 1:1 to about 1:10; or about 1:2 to about 1:9; or about 1:3 to about 1:8; or about 1:3 to about 1:7; or about 1:3 to about 1:5, or about 1:5 to about 1:6; and is optionally about 1:4.

33. The lipid nanoparticle composition of any one of claims 30 to 32, wherein the molar ratio of the structural lipid over the PEG lipid is in the range of about 50:1 to about 1:0.025; or about 40:1 to about 5:1: or about 40:1 to about 10:1; or about 34:1 to about 30:1, or about 30:1 to about 15:1: or about 30:1 to about 20:1; and is optionally about 50:1.5.

34. The lipid nanoparticle composition of claim 30, wherein the molar ratio of the compound of Formula 1, the phospholipid, the structural lipid, and the PEG lipid is about 50: about 10: about 38.5: about 1.5 respectively.

35. The lipid nanoparticle composition of claim 30, wherein the molar ratio of the compound of Formula (I), the phospholipid, the structural lipid, and the PEG lipid is about 31.3: about 8.2: about 60: about 0.5 respectively.

36. The lipid nanoparticle composition of claim 30, wherein the molar ratio of the compound of Formula (I), the phospholipid, the structural lipid, and the PEG lipid is about 23.4: about 16.1: about 60: about 0.5 respectively.

37. The lipid nanoparticle composition of claim 30, wherein the molar ratio of the compound of Formula (I), the phospholipid, the structural lipid, and the PEG lipid is about 31.3: about 8.2: about 59: about 1.5 respectively.

38. The lipid nanoparticle composition of any one of claims 1 to 37, wherein the lipid nanoparticles in the composition encapsulate one or more therapeutic, prophylactic, or diagnostic agents.

39. The lipid nanoparticle composition of claim 38, wherein the lipid nanoparticles encapsulate one or more polynucleotides.

40. The lipid nanoparticle composition of claim 39, wherein the polynucleotide is RNA.

41. The lipid nanoparticle composition of claim 40, wherein the RNA is one or more selected from a small RNA, ribozyme, small interfering RNA (siRNA), asymmetrical interfering RNA (aiRNA), microRNA (miRNA), Dicer-substrate RNA, double stranded RNA (dsRNA), small hairpin RNA (shRNA), transfer RNA (tRNA), messenger RNA (mRNA), double stranded mRNA, circular RNA (ORNA), and self-amplifying mRNA (SAM).

42. The lipid nanoparticle composition of claim 41, wherein the RNA is mRNA or modified mRNA (mmRNA).

43. The lipid nanoparticle composition of claim 42, wherein the mRNA or mmRNA encodes polypeptide of an infectious organism, which is optionally a virus, and which is optionally a betacoronavirus, an influenza virus, or a herpesvirus.

44. The lipid nanoparticle composition of claim 42, wherein the mRNA or mmRNA encodes one or more cancer associated polypeptide, epitope, antigen, or neoantigen.

45. The lipid nanoparticle composition of claim 39, wherein the polynucleotide is an antisense oligonucleotide.

46. The lipid nanoparticle composition of claim 39, wherein the polynucleotide is DNA.

47. The lipid nanoparticle composition of any one of claims 39 to 46, wherein the ratio between the amines of the ionizable lipid of Formula 1 and phosphates of the polynucleotide (N: P ratio) is from about 2:1 to about 50:1, or about 2:1 to about 40:1, or about 2:1 to about 20:1, or about 2:1 to about 15:1, or about 2:1 to about 12:1, or about 35:1 to about 45:1, or about 2:1 to about 10:1, or about 3:1 to about 12:1, or about 4:1 to about 12:1, or about 5:1 to about 7:1.

48. The lipid nanoparticle composition of claim 47, wherein the N: P ratio is about 6:1 or about 12:1.

49. The lipid nanoparticle composition of any one of claim 30 to claim 48 wherein the molar ratio of the compound of Formula 1, the phospholipid, the structural lipid, and the PEG lipid is about 23.4 to about 50: about 8.2 to about 10: about 38.5 to about 60: about 0.5 to about 1.5.

50. A method of delivering a therapeutic agent, the method comprising administering to a subject in need thereof the composition of any one of claims 1 to 49.

51. The method of claim 50, wherein the subject is a mammal, and optionally a human.

52. The method of claim 50, wherein the subject is a bird.

53. The method of claim 50, 51, or 52, wherein the composition is administered intravenously, intramuscularly, intradermally, subcutaneously, intranasally, or by inhalation.
